(19) United States
(12) Patent Application Publication
(10) Pub. No.: US 2021/0276971 A1
(43) Pub. Date: Sep. 9, 2021

(54) TRIAZAMACROCYCLE-DERIVED CHELATOR COMPOSITIONS FOR COORDINATION OF IMAGING AND THERAPY METAL IONS AND METHODS OF USING SAME

(71) Applicant: The Research Foundation for The State University of New York, Albany, NY (US)

(72) Inventors: Eszter Boros, Stony Brook, NY (US); Brett Vaughn, Centereach, NY (US)

(73) Assignee: The Research Foundation for The State University of New York, Albany, NY (US)

(21) Appl. No.: 17/253,307

(22) PCT Filed: Jun. 20, 2019
(86) PCT No.: PCT/US2019/038318

§ 371 Date: Dec. 17, 2020

Related U.S. Application Data
(60) Provisional application No. 62/687,581, filed on Jun. 20, 2018.

(51) Int. Cl. C07D 401/06; A61K 51/04

(52) U.S. Cl. CPC C07D 401/06; A61K 51/0482

(57) ABSTRACT

The present invention provides a compound having the structure:

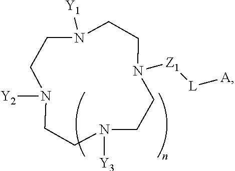

and methods of using the compound in targeted PET imaging.

This application claims priority of U.S. Provisional Application No. 62/687,581, filed Jun. 20, 2018, the contents of which are hereby incorporated by reference.

### GOVERNMENT SUPPORT

This invention was made with government support under grant number HL127522 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Prostate cancer is the most common cancer in Europe and the United States amongst men. Typically early detection allows for successful treatment of disease, however, prognosis after the occurrence of metastatic disease is poor.¹ Thus, incorporation of early detection methods into typical health regiments of the aging male population has become the mainstay in developed countries, such as frequent testing of serum prostate specific antigen (PSA) levels. In case of elevated PSA values (>4 ng/mL), patients are further assessed via MRI-assisted biopsy, an invasive and costly procedure causing extensive patient discomfort and identifying over 50V of cases with an elevated PSA as false positives.

While early detection of non-metastatic prostate cancer provides almost a 100% 5-year survival, the prognosis of recurrent, metastatic and castrate resistant disease is poor. Currently, no curative options exist for patients with metastatic castration-resistant prostate cancer (mCRPC).² The potential of new isotope-based pharmaceuticals for symptom relief and/or prolongation of survival has been recognized; a number of tracers have been developed for this purpose. The trans membrane protein prostate specific membrane antigen (PSMA) has recently emerged as an attractive imaging and therapy target in prostate cancer. One radiolabeled probe targeting PSMA has been FDA approved. The monoclonal antibody ¹¹¹In-capromab (ProstaScint) was initially developed as a PSMA-specific SPECT imaging tracer.³⁻⁵ ProstaScint obtained FDA-approval but showed poor clinical feasibility due to inefficient tracer uptake through targeting of the intracellular domain of PSMA.

Structure-activity studies on small molecule inhibitors for the large extracellular domain of PSMA revealed that glutamate-urea-glutamate-based small molecules bearing a 2-[3-(1,3-dicarboxypropyl)-ureido]pentanedioic acid (DUPA)⁶ served as mimics of the endogenous substrate N-acetyl-L-aspartyl-L-glutamate (NAAG) and could target and inhibit the catalytic active site.^{7,8 18}F-DCFPyL, an ¹⁸F-labeled is undergoing phase III clinical trials.⁹⁻¹¹) The primary drawback of this compound is the lack of radiotherapeutic analogue as clinical management tool after diagnosis with the imaging tracer. There is a clear, unmet need for improved, non-invasive staging tools for prostate cancer to improve screening and reduce false positives, as well as dual, theranostic tracers that provide both an imaging and a therapeutic tool for improved treatment of incurable mCRPC prostate cancer.

⁴⁴Scandium is an ideal short-lived radioisotope with a half-life well matched to the typical pharmacokinetics of small molecules, peptides and small biologics and with ideal emission properties (t_{1/2}=3.97 h, Eₘₑₐₙ β⁺=632 keV) for PET imaging. The isotope ⁴⁷Sc, a low-energy β⁻ emitter (t_{1/2}=80.4 h, Eₘₑₐₙ β⁻=162 keV) is an isotope with identical chemical properties to ⁴⁴Sc and highly suited for radiotherapeutic applications.¹² The synthesis of ⁴⁴Sc can be achieved using both a cyclotron as well as a generator source; proton-irradiation of a ⁴⁴Ca target¹³ as well as the elution of a ⁴⁴Ti generator both produce the isotope with high specific activity.¹⁴ A first ⁴⁴Sc-tracer targeting PSMA has been evaluated but requires heating at 95° C. to obtain non-quantitative radiolabeling.¹⁵

### SUMMARY OF THE INVENTION

The present invention provides a compound having the structure:

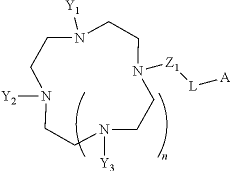

wherein
n is 0 or 1;
Y₁, Y₂ and Y₃ are each, independently, —H, alkylheteroaryl, alkyl-CO₂H, alkylaryl-CO₂H, alkylheteroaryl-CO₂H, alkyl-CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH) (alkyl-CO₂H), alkyl-N(alkylheteroaryl-OH) (alkyl-CO₂H), alkyl-P(O) (OH)₂, alkylaryl-P(O) (OH)₂ and alkylheteroaryl-P(O) (OH)₂,
   wherein each occurrence of R₄ is, independently, —H, alkyl, alkenyl, alkynyl, alkyl-aryl, alkyl-heteroaryl, aryl, heteroaryl, alkyl-CF₃ or —Si(alkyl)₃;
Z₁ is

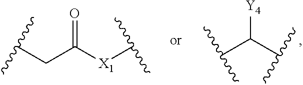

wherein X₁ is NH, O or S, and
Y₄ is —CO₂H, —CO₂R₅, aryl-CO₂H, heteroaryl-CO₂H, aryl-CO₂R₅ or heteroaryl-CO₂R₅,
   wherein each occurrence of R₅ is, independently, —H, alkyl, alkenyl, alkynyl, alkyl-aryl, alkyl-heteroaryl, aryl, heteroaryl, alkyl-CF₃ or —Si (alkyl) 3;
A is a targeting moiety; and
L is a chemical linker,

or a pharmaceutically acceptable salt of the compound.

### BRIEF DESCRIPTION OF THE FIGURES

FIG. 1: Time-dependent complexation at 10 nmol at 10, 30, and 60 min time-points of various ligands.

FIG. 2A: Initial chelators screened for radiolabeling properties.

FIG. 2B: Results of the initial chelator screen identify monopic as a chelator with ideal ⁴⁴Sc-radiolabeling properties.

FIG. 3: Comparative, crude radiolabeling traces obtained with ⁴⁴Sc show the rapid and clean complexation with monopic after 10 minutes at 80° C. (1 nmol ligand). Under identical conditions, DOTA does not result in quantitative radiolabeling.

FIG. 4A: ¹H NMR studies of Sc-complexes formed with EDTA, DOTA and Monopic reveal that the Sc(monopic) and Lu(monopic) complexes exhibit slow structural isomerism and efficiently shield the metal ion from solvent molecules. *denotes ¹H-signals from residual, uncomplexed ligand in solution.

FIG. 4B: ⁴⁵Sc NMR studies of Sc-complexes formed with EDTA, DOTA and Monopic reveal that the Sc(monopic) and Lu(monopic) complexes exhibit slow structural isomerism and efficiently shield the metal ion from solvent molecules.

FIG. 5: Chemical synthesis of protected monopic precursor 3a and the functionalized conjugate picaga-DUPA.

FIG. 6A: Characterization of non-interconverting diastereomers formed upon complexation with picaga-DUPA; coordinating water and the DUPA-conjugate are omitted from the structural drawing for clarity.

FIG. 6B: Radiolabeling yields obtained in dependence of increasing amounts of ligand used with 100 μCi ⁴⁴Sc per labelling attempt.

FIG. 7: Coronal PET-CT slices 90 minute post injection show the superior image quality achieved with ⁴⁴Sc-picaga-DUPA, confirmed by the results obtained from the biodistribution analysis 120 min post injection.

FIG. 8: Cell binding assay of DO2APic-DUPA, NOPic-DUPA And NO2APic-DUPA.

FIG. 9: Biodistribution indicates enhanced, target specific accumulation of ⁶⁴Cu(DO2Apic)-DUPA, and ⁶⁴Cu (NOpic-DUPA) in PSMA− expressing xenografts

FIG. 10: Chelators for scandium and copper isotopes.

FIG. 11: Imaging probes containing chelator and targeting moiety.

FIG. 12: DOTA and PICAGA formulation check.

FIG. 13: Sc-Overlay and Lu-Overlay—A stock solution of PICAGA (2 mg/mL) in DI water was prepared. 1.1 equivalents of metal added to the ligand. ScCl₃.6H₂O or Lu(ClO₄)₃.6H₂O. pH was adjusted to 6-7 with 5 M NaOH. Complex solutions were injected on HPLC. Each peak associated with the PICAGA (5.7 and 5.9 min) was collected and reinjected at 20 min. Each peak demonstrated limited interconversion

### DETAILED DESCRIPTION OF THE INVENTION

The present invention provides a compound having the structure:

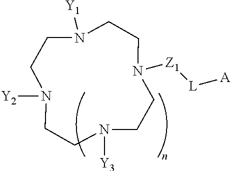

wherein
n is 0 or 1;
Y₁, Y₂ and Y₃ are each, independently, —H, alkylheteroaryl, alkyl-CO₂H, alkylaryl-CO₂H, alkylheteroaryl-CO₂H, alkyl-CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N (alkylaryl-OH) (alkyl-CO₂H), alkyl-N(alkylheteroaryl-OH) (alkyl-CO₂H), alkyl-P(O) (OH)₂, alkylaryl-P(O) (OH)₂ and alkylheteroaryl-P(O) (OH)₂,
   wherein each occurrence of R₄ is, independently, —H, alkyl, alkenyl, alkynyl, alkyl-aryl, alkyl-heteroaryl, aryl, heteroaryl, alkyl-CF₃ or —Si(alkyl)₃;
Z₁ is

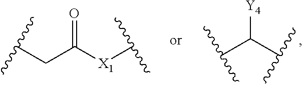

wherein X₁ is NH, O or S, and
Y₄ is —CO₂H, —CO₂R₅, aryl-CO₂H, heteroaryl-CO₂H, aryl-CO₂R₅ or heteroaryl-CO₂RP,
   wherein each occurrence of R₅ is, independently, —H, alkyl, alkenyl, alkynyl, alkyl-aryl, alkyl-heteroaryl, aryl, heteroaryl, alkyl-CF₃ or —Si (alkyl) s;
A is a targeting moiety; and
L is a chemical linker,

or a pharmaceutically acceptable salt of the compound.

In some embodiments, the compound wherein the targeting moiety A is a moiety with specificity for a target protein on the surface of a cell.

In some embodiments, the compound wherein the targeting molecule A is a moiety with specificity for a target antigen on the surface of a cell.

In some embodiments, the compound wherein the targeting moiety A is a small molecule, a peptide or an antibody or a derivative or fragment thereof.

In some embodiments, the compound wherein the targeting moiety A is trastuzumab, bombesin, somatostatin or 2-[3-(1,3-dicarboxypropyl)ureido]pentanedioic acid (DUPA) or a derivative or fragment thereof.

In some embodiments, the compound wherein the targeting moiety A is covalently attached to the chemical linker L.

In some embodiments, the compound wherein the bond between the targeting moiety A and the chemical linker L is formed by reacting a first terminal reactive group on the targeting moiety A with a second terminal reactive group on the chemical linker L.

In some embodiments, the compound wherein the bond between the imaging moiety and the chemical linker L is formed by reacting a first terminal reactive group on the imaging moiety with a second terminal reactive group on the chemical linker L.

In some embodiments, the compound wherein the bond between the targeting moiety A and the chemical linker L is formed by reacting a carboxylic acid moiety on the targeting moiety A with an amine moiety on the chemical linker L.

In some embodiments, the compound wherein the chemical linker L is an alkyl, alkenyl, alkynyl, alkylether, alkylthioether, alkylamino, alkylamido, alkylester, alkylaryl, alklyheteroaryl, aryl, heteroaryl, a natural amino acid, an unnatural amino acid, a disulfide or thioether containing linker or combinations thereof.

In some embodiments, the compound wherein the chemical linker L is a releasable linker.

In some embodiments, the compound wherein the chemical linker L is a non-releasable linker.

In some embodiments, the compound having the structure:

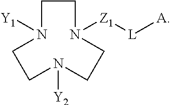

In some embodiments, the compound wherein
Z₁ is

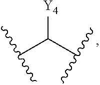

wherein Y₄ is —CO₂H, -aryl-CO₂—H or heteroaryl-CO₂H.

In some embodiments, the compound wherein
Y₄ is —CO₂H.

In some embodiments, the compound wherein
Z₁ is,

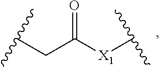

wherein X₁ is NH.

In some embodiments, the compound wherein
Y₁ and Y₂ are each, independently, —H, alkyl-CO₂H, alkylaryl-CO₂H or alkylheteroaryl-CO₂H.

In some embodiments, the compound wherein
one of Y₁ or Y₂ is —H, or
each of Y₁ and Y₂ is —H.

In some embodiments, the compound wherein
one of Y₁ or Y₂ is alkyl-CO₂H, or
each of Y₁ and Y₂ is alkyl-CO₂H.

In some embodiments, the compound wherein
one of Y₁ or Y₂ is alkylaryl-CO₂H or alkylheteroaryl-CO₂H, or
each of Y₁ and Y₂ are alkylaryl-CO₂H or alkylheteroaryl-CO₂H.

In some embodiments, the compound wherein the heteroaryl is a pyridyl.

In some embodiments, the compound having the structure:

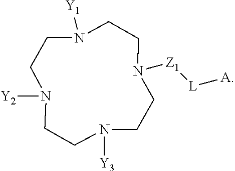

In some embodiments, the compound wherein
Z₁ is

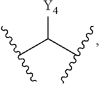

wherein Y₄ is —CO₂H, -aryl-CO₂H or heteroaryl-CO₂H.

In some embodiments, the compound wherein
Y₄ is —CO₂H.

In some embodiments, the compound wherein
Z₁ is

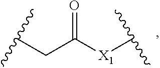

wherein X₁ is NH.

In some embodiments, the compound wherein
Y₁, Y₂ and Y₃ are each, independently, —H, alkyl-CO₂H,
alkylaryl-CO₂H or alkylheteroaryl-CO₂H.

In some embodiments, the compound wherein
one of Y₁, Y₂ or Y₃ is —H, or
two of Y₁, Y₂ or Y₃ are —H, or
each of Y₁, Y₂, and Y₃ is —H.

In some embodiments, the compound wherein
one of Y₁, Y₂ or Y₃ is alkyl-CO₂H, or
two of Y₁, Y₂ or Y₃ are alkyl-CO₂H, or
each of Y₁, Y₂ and Y₃ is alkyl-CO₂H.

In some embodiments, the compound wherein
one of Y₁, Y₂ or Y₃ is alkylaryl-CO₂H or alkylheteroaryl-CO₂H, or
two of Y₁, Y₂ or Y₃ is alkylaryl-CO₂H or alkylheteroaryl-CO₂H, or
each of Y₁, Y₂ and Y₃ are alkylaryl-CO₂H or alkylheteroaryl-CO₂H.

In some embodiments, the compound wherein the heteroaryl is pyridyl.

In some embodiments, the compound a pharmaceutical composition comprising the compound of the present invention and a pharmaceutically acceptable carrier.

In some embodiments, a metal complex comprising the compound of the present invention, wherein the compound coordinates or chelates or complexes to a metal.

In some embodiments, the metal complex wherein the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal complex wherein the metal is Scandium-47 (⁴⁷Sc) or Copper-67 (⁶⁷Cu).

In some embodiments, the metal complex having the structure:

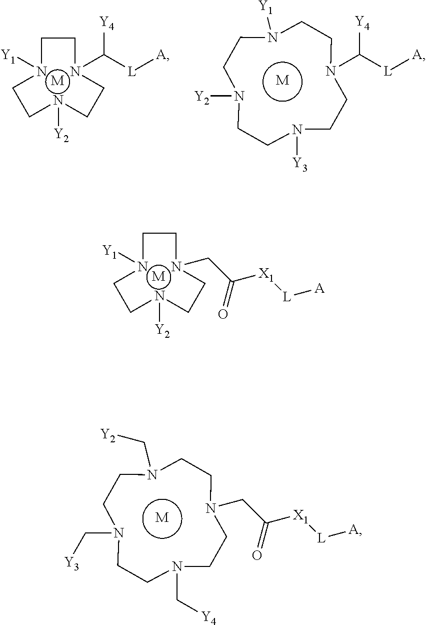

or a pharmaceutically salt thereof.

In some embodiments, a pharmaceutical composition comprising the metal complex of the present invention and a pharmaceutically acceptable carrier.

The present invention provides a method of detecting target cells in a subject comprising administering an effective amount of the metal complex of the present invention or the composition of the present invention to the subject, and imaging the subject with a molecular imaging device to detect the metal complex or composition in the subject.

In some embodiments, the method wherein the compound or composition specifically accumulates at the target cells

In some embodiments, the method wherein the target cells are cancer cells.

In some embodiments, the method wherein the target cells are prostate cancer cells.

In some embodiments, the method wherein detection of the compound or composition in the target cells of the subject is an indication that cancers cells are present in subject.

In some embodiments, the method wherein the compound or composition is detected using a PET imaging device.

The present invention provides a method of imaging target cells in a subject comprising:
1) administering to the subject an effective amount of the metal complex of the present invention or a pharmaceutically acceptable salt thereof, or the composition of the present invention,
   wherein the compound specifically accumulates at the target cells in the subject;
3) detecting in the subject the location of the metal complex or the composition; and
4) obtaining an image of the target cells in the subject based on the location of the metal complex or the composition in the subject.

In some embodiments, the method wherein the compound or composition is detected using a PET imaging device.

In some embodiments, the method wherein the image obtained is a three-dimensional image.

The present invention provides a method of detecting the presence of target cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the target cells based on the amount of the metal complex or composition determined to be present in the subject.

In some embodiments, the method wherein the detecting is performed by a Positron Emission Tomography (PET) device.

In some embodiments, the method further comprising quantifying the amount of the compound in the subject and comparing the quantity to a predetermined control.

In some embodiments, the method further comprising determining whether the subject is afflicted with cancer based on the amount of the compound in the subject.

In some embodiments, the method further comprising determining the stage of the cancer.

The present invention provides a method of reducing the size of a tumor or of inhibiting proliferation of cancer cells comprising contacting the tumor or cancer cells with the metal complex of the present invention or a pharmaceutically acceptable salt thereof, or the composition of the present invention, so as to thereby reducing the size of the tumor or inhibit proliferation of the cancer cells.

In some embodiments, the compound wherein A has the structure:

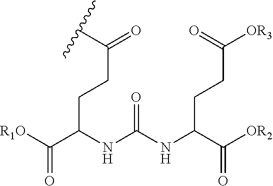

wherein
R₁, R₂ and R₃ are each, independently, —H, alkyl, alkenyl, alkynyl, alkyl-aryl, alkyl-heteroaryl, aryl, heteroaryl, alkyl-CF₃ or —Si(alkyl)₃.

In some embodiments, the compound having the structure:

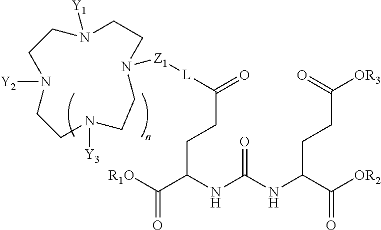

wherein
n is 0 or 1;
R₁, R₂ and R₃ are each, independently, —H, alkyl, alkenyl, alkynyl, alkyl-aryl, alkyl-heteroaryl, aryl, heteroaryl, alkyl-CF₃ or —Si(alkyl)₃;
Y₁, Y₂ and Y₃ are each, independently, —H, alkylheteroaryl, alkyl-CO₂H, alkylaryl-CO₂H, alkylheteroaryl-CO₂H, alkyl-CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH) (alkyl-CO₂H), alkyl-N(alkylheteroaryl-OH) (alkyl-CO₂H), alkyl-P(O) (OH)₂, alkylaryl-P(O) (OH)₂, alkylheteroaryl-P(O) (OH)₂,
   wherein each occurrence of R₄ is, independently, —H, alkyl, alkenyl, alkynyl, alkyl-aryl, alkyl-heteroaryl, aryl, heteroaryl, alkyl-CF₃ or —Si(alkyl)₃;
Z₁ is

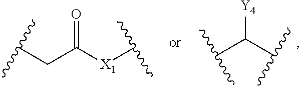

wherein X₁ is NH, O or S, and
Y₄ is —CO₂H, —CO₂R₅, aryl-CO₂—H, heteroaryl-CO₂H, aryl-CO₂R₅ or heteroaryl-CO₂R₅,
   wherein each occurrence of R₅ is, independently, —H, alkyl, alkenyl, alkynyl, alkyl-aryl, alkyl-heteroaryl, aryl, heteroaryl, alkyl-CF₃ or —Si (alkyl)₃; and
L is a chemical linker,

or a pharmaceutically acceptable salt of the compound.

In some embodiments, the compound having the structure:

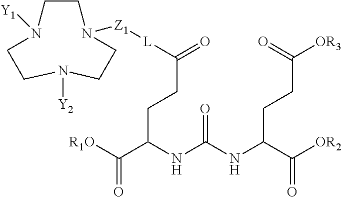

or a pharmaceutically acceptable salt of the compound.

In some embodiments, the compound wherein
Z₁ is

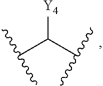

wherein Y₄ is —CO₂H, —CO₂R₅, aryl-CO₂H, heteroaryl-CO₂H, aryl-CO₂R₅ or heteroaryl-CO₂R₅,
   wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃.

55. The compound of claim 54, wherein
Y₄ is —CO₂H, aryl-CO₂H or heteroaryl-CO₂H.

In some embodiments, the compound wherein Z₁ is 1

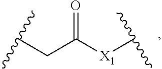

wherein X₁ is NH, O or S.

In some embodiments, the compound wherein X; is NH.

In some embodiments, the compound wherein Y₁ and Y₂ are each, independently, —H, alkyl-CO₂H, alkylaryl-CO₂H or alkylheteroaryl-CO₂H.

In some embodiments, the compound wherein one of Y₁ or Y₂ is —H, or each of Y₁ and Y₂ is —H.

In some embodiments, the compound wherein one of Y₁ or Y₂ is alkyl-CO₂H, or each of Y₁ and Y₂ is alkyl-CO₂H.

In some embodiments, the compound wherein one of Y₁ or Y₂ is alkylaryl-CO₂H or alkylheteroaryl-CO₂H, or each of Y₁ and Y₂ are alkylaryl-CO₂H or alkylheteroaryl-CO₂H.

In some embodiments, the compound wherein the heteroaryl is pyridyl.

In some embodiments, the compound having the structure:

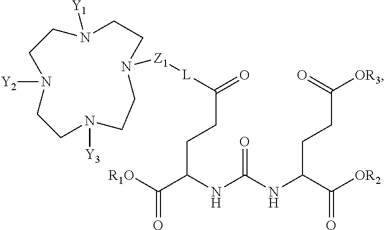

or a pharmaceutically acceptable salt of the compound.

In some embodiments, the compound wherein
Z₁ is

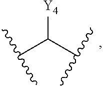

Y₄ is —CO₂H, —CO₂R₅, aryl-CO₂H, heteroaryl-CO₂H, aryl-CO₂R₅ or heteroaryl-CO₂R₅,
   wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃.

In some embodiments, the compound wherein Y₄ is —CO₂H, aryl-CO₂H or heteroaryl-CO₂H.

In some embodiments, the compound wherein
Z₁ is

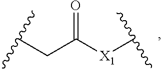

wherein X₁ is NH, O or S.

In some embodiments, the compound wherein X₂ is NH.

In some embodiments, the compound wherein Y₁, Y₂ and Y₃ are each, independently, —H, alkyl-CO₂H, alkylaryl-CO₂H or alkylheteroaryl-CO₂H.

In some embodiments, the compound wherein one of Y₁, Y₂ or Y₃ is —H, or two of Y₁, Y₂ or Y₃ are —H, or each of Y₁, Y₂ and Y₃ is —H.

In some embodiments, the compound wherein one of Y₁, Y₂ or Y₃ is alkyl-CO₂H, or two of Y₁, Y₂ or Y₃ are alkyl-CO₂H, or each of Y₁, Y₂ and Y₃ is alkyl-CO₂H.

In some embodiments, the compound wherein one of Y₁, Y₂ or Y₃ is alkylaryl-CO₂H or alkylheteroaryl-CO₂H, or
two of Y₁, Y₂ or Y₃ is alkylaryl-CO₂H or alkylheteroaryl-CO₂H, or
each of Y₁, Y₂ and Y₃ are alkylaryl-CO₂H or alkylheteroaryl-CO₂H.

In some embodiments, the compound wherein the heteroaryl is pyridyl.

In some embodiments, the compound wherein R₁, R₂ and R₃ are each H.

In some embodiments, the compound wherein the chemical linker L is an alkyl, alkenyl, alkynyl, alkylether, alkylthioether, alkylamino, alkylamido, alkylester, alkylaryl, alklyheteroaryl, aryl, heteroaryl, a natural amino acid, an unnatural amino acid, a disulfide or thioether containing linker or combinations thereof.

In some embodiments, the compound wherein the chemical linker L is alkyl, alkenyl, alkynyl, alkyl-O-alkyl, alkyl-O-alkyl-O-alkyl, alkyl-NH, alkyl-NH-alkyl, alkyl-C(O) O-alkyl, alkyl-OC(O)-alkyl alkyl-CO-alkyl, alkyl-C(O)NH-alkyl, alkyl-NHC(O)-alkyl or alkyl-C(O)NH-alkyl-NH or combinations thereof.

In some embodiments, the compound wherein chemical linker L a C₂-C₁₂ alkyl, C₂-C₁₂ alkyl-NH, C₂-C₁₂ alkyl-NHC(O)—C₂-C₁₂ alkyl, C₂-C₁₂ alkyl-C(O) NH—C₂-C₁₂ alkyl or C₁-C₁₂ alkyl-C(O) NH—C₁-C₁₂ alkyl-NH.

In some embodiments, the compound having the structure:

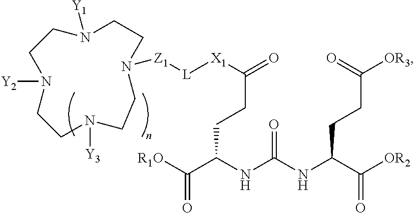

or a pharmaceutically acceptable salt of the compound.

In some embodiments, the compound having the structure:

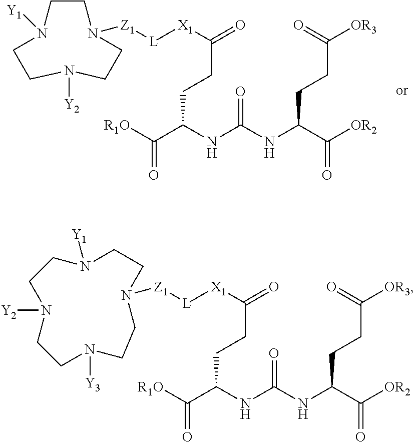

or a pharmaceutically acceptable salt of the compound.

In some embodiments, the compound having the structure:

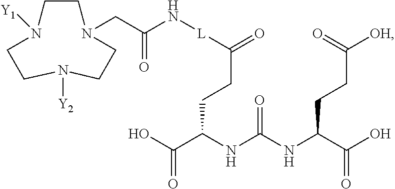

wherein
Y₁ is —H,

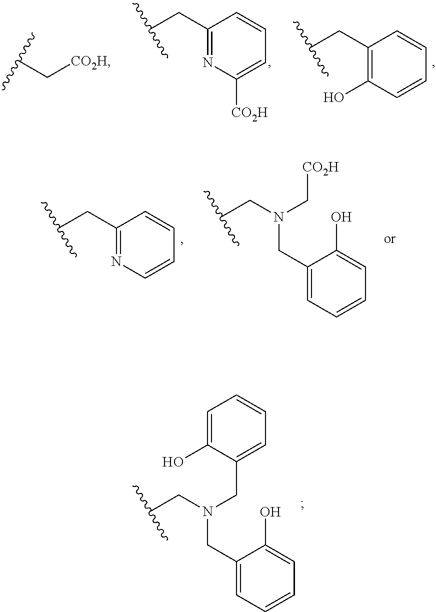 [SMILES: *CC(=O)O ; *Cc1cccc(C(=O)O)n1 ; *Cc1ccccc1O ; *Cc1ccccn1 ; *CN(CC(=O)O)Cc1ccccc1O ; *CN(Cc1ccccc1O)Cc1ccccc1O]

Y₂ is —H,

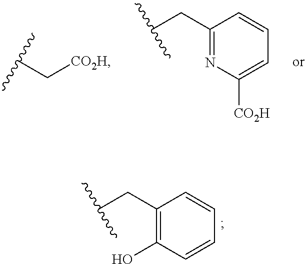 [SMILES: *CC(=O)O ; *Cc1cccc(C(=O)O)n1 ; *Cc1ccccc1O]

L is alkyl-NH,

or a pharmaceutically acceptable salt of the compound.

In some embodiments, the compound wherein L is C₄-alkyl-NH.

In some embodiments, the compound, wherein L is C₅-alkyl-NH.

In some embodiments, the compound having the structure:

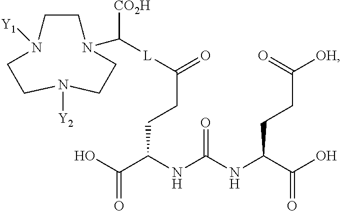

wherein
Y₁ is —H,

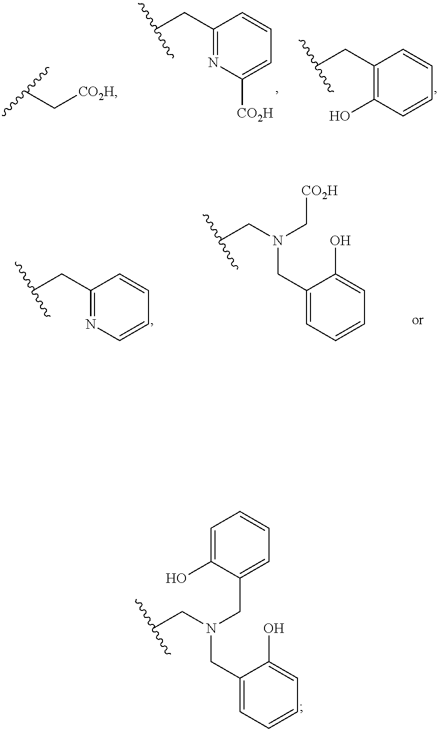

Y₂ is —H,

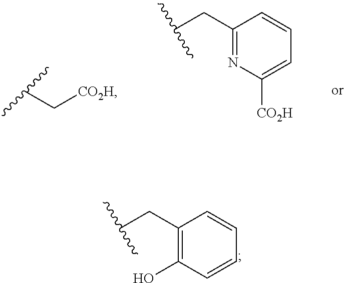 [SMILES: *CC(=O)O ; *Cc1cccc(C(=O)O)n1 ; "or" ; *Cc1ccccc1O ; ";"]

L is alkyl-C(O)NH-alkyl-NH,

or a pharmaceutically acceptable salt of the compound.

In some embodiments, the compound wherein L is a C₂-alkyl-C(O)NH—C₄ alkyl-NH or C₂-alkyl-C(O)NH—C₅ alkyl-NH.

In some embodiments, the compound having the structure:

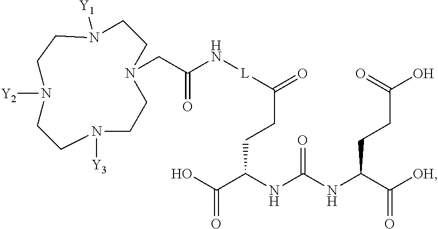

wherein
Y₁ is —H,

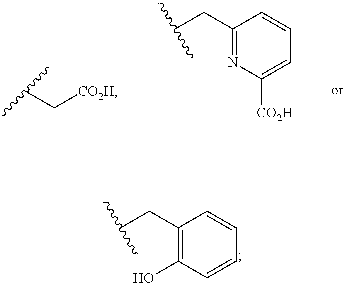 [SMILES: *CC(=O)O ; *Cc1cccc(C(=O)O)n1 ; "or" ; *Cc1ccccc1O ; ";"]

Y₂ is —H,

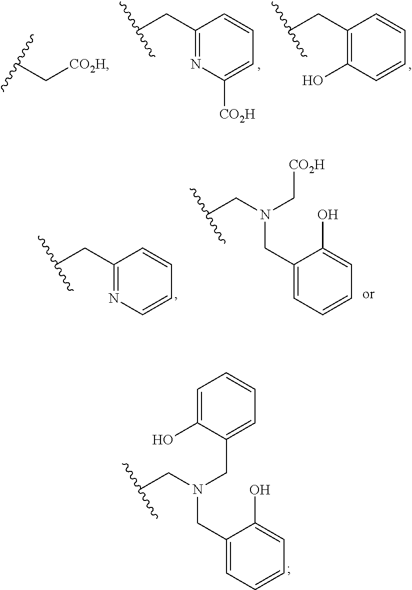

Y₃ is —H,

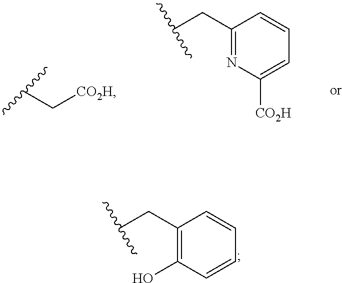 [SMILES: *CC(=O)O ; *Cc1cccc(C(=O)O)n1 ; "or" ; *Cc1ccccc1O ; ";"]

and
L is alkyl-NH,

or a pharmaceutically acceptable salt of the compound.

In some embodiments, the compound wherein L is a C₄-alkyl-NH or C₅-alkyl-NH.

In some embodiments, the compound having the structure:

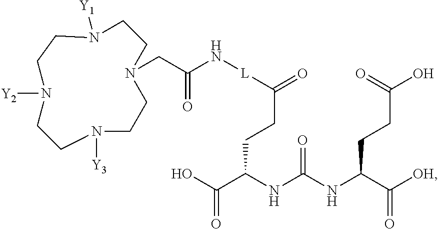

wherein
Y₁ is —H,

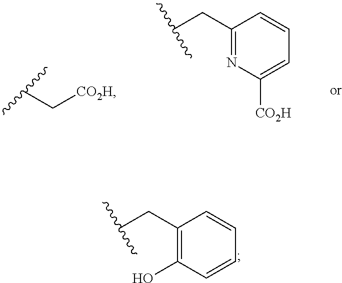 [SMILES: *CC(=O)O ; *Cc1cccc(C(=O)O)n1 ; "or" ; *Cc1ccccc1O ; ";"]

Y₂ is —H,

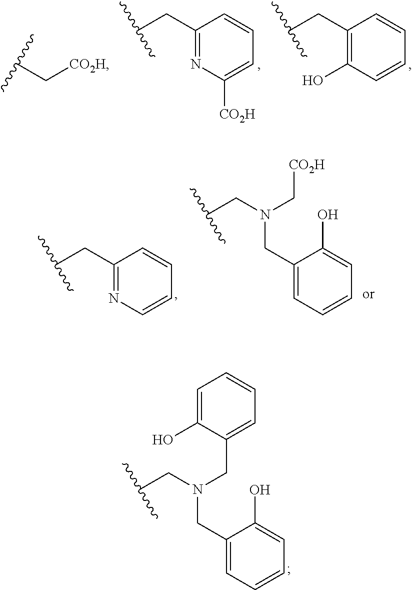

Y₃ is —H,

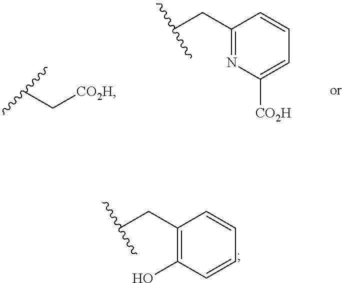 [SMILES: *CC(=O)O ; *Cc1cccc(C(=O)O)n1 ; "or" ; *Cc1ccccc1O ; ";"]

L is an alkyl-C(O)NH-alkyl chemical linker,

or a pharmaceutically acceptable salt of the compound.

In some embodiments, the compound wherein L is C₂-alkyl-C(O)NH—C₄ alkyl-NH or C₂-alkyl-C(O)NH—C₅ alkyl-NH.

In some embodiments, the compound wherein each of Y₁ and Y₃ is

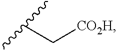 [SMILES: *CC(=O)O]

In some embodiments, the compound having the structure:

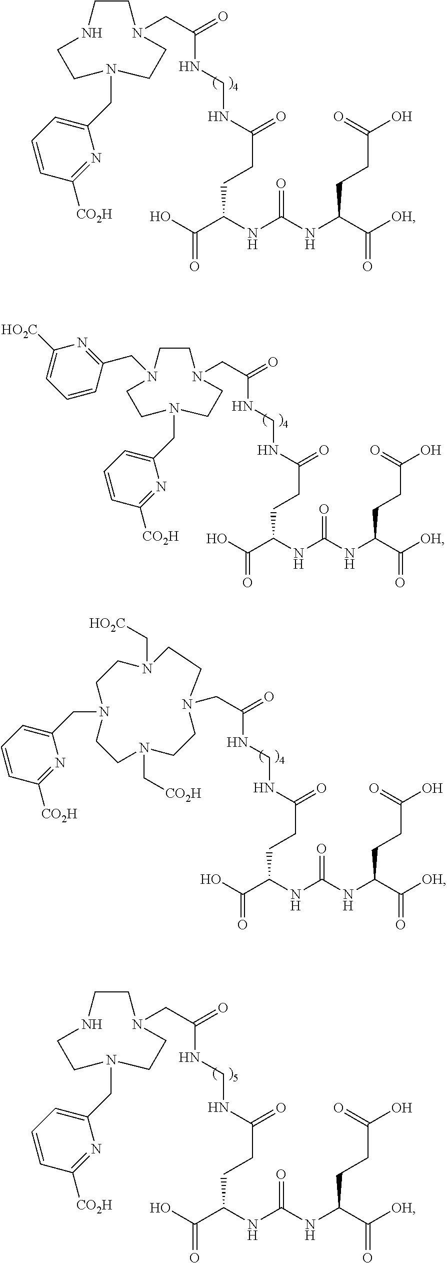

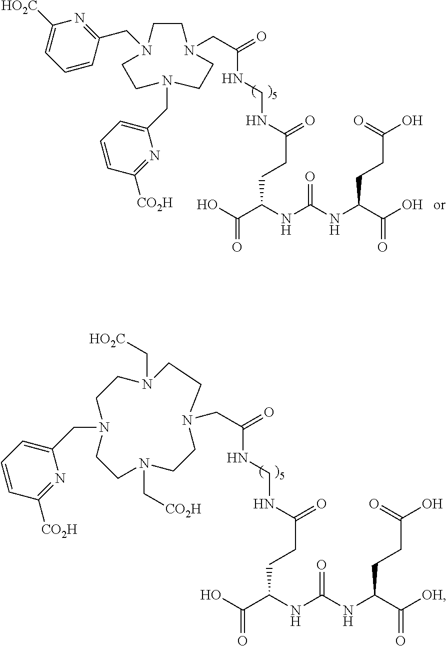

or a pharmaceutically salt thereof.

In some embodiments, the compound having the structure:

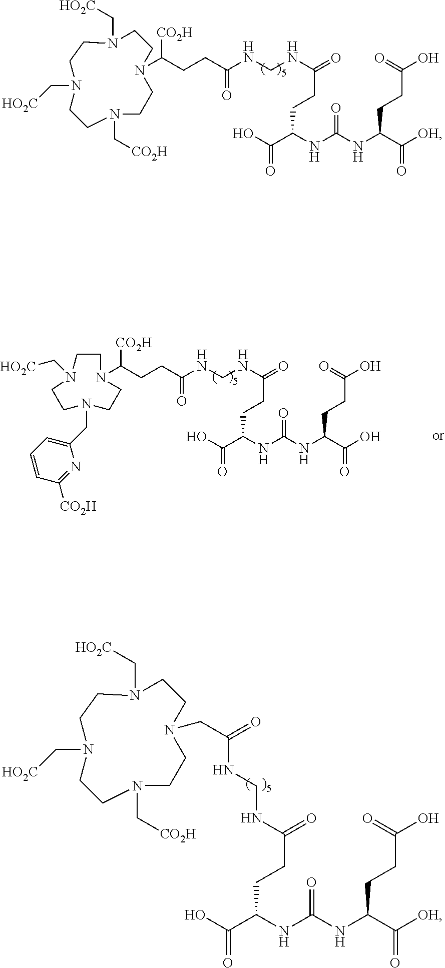

or a pharmaceutically salt thereof.

In some embodiments, a pharmaceutical composition comprising the compound of the present invention and a pharmaceutically acceptable carrier.

In some embodiments, a metal complex comprising the compound of the present invention, wherein the compound coordinates to a metal.

In some embodiments, the metal complex wherein the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium 177 (⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal complex wherein the metal is Scandium-47 (⁴⁷Sc) or Copper-67 (⁵⁷Cu).

In some embodiments, the metal complex having the structure:

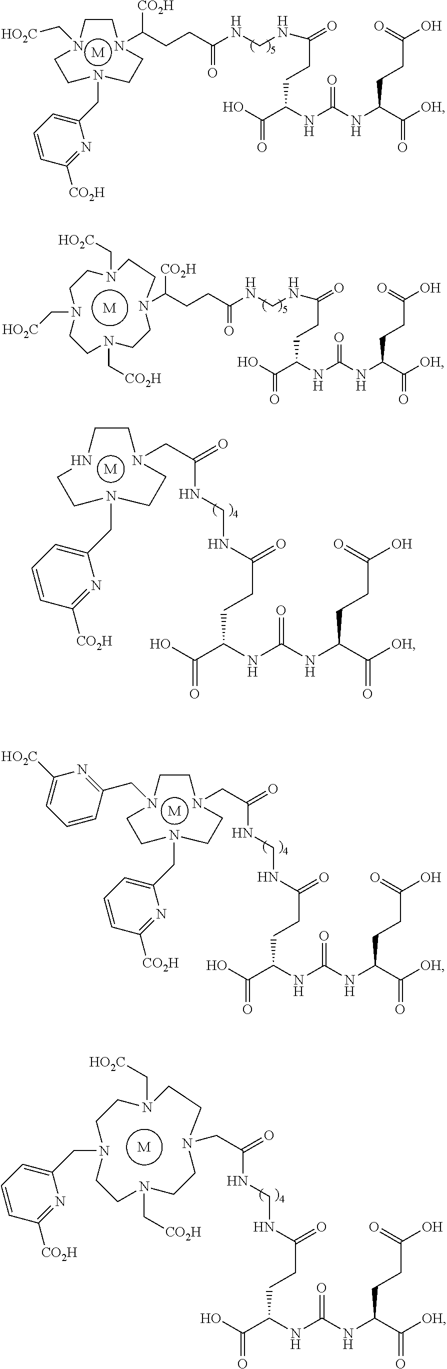

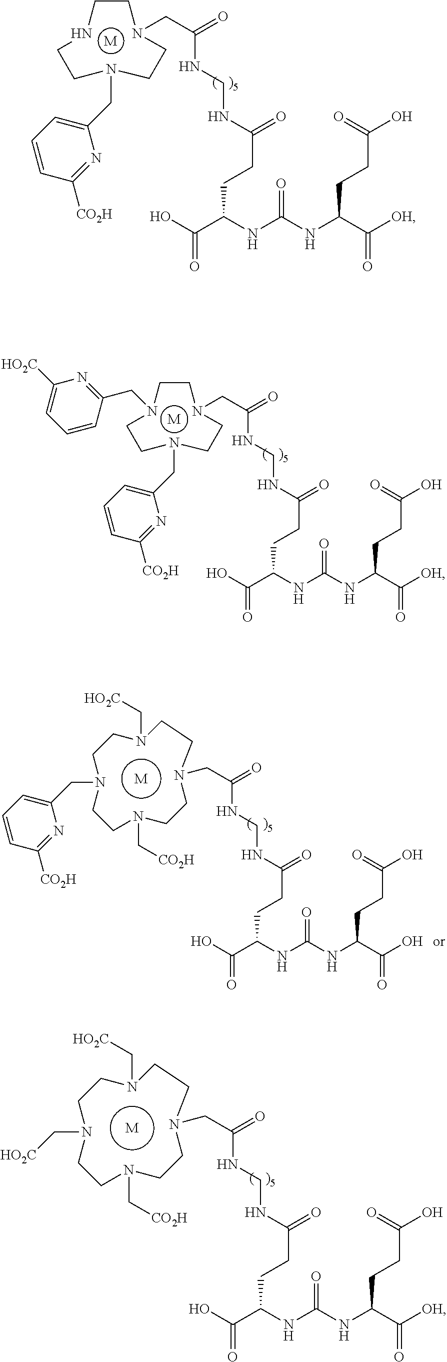

or a pharmaceutically salt thereof.

In some embodiments, a pharmaceutical composition comprising the metal complex of the present invention and a pharmaceutically acceptable carrier.

The present invention provides a method of detecting cancer cells in a subject comprising administering an effective amount of the metal complex of the present invention or the composition of the present invention to the subject, and imaging the subject with a molecular imaging device to detect the metal complex or composition in the subject.

In some embodiments, the method wherein the cancer cells are prostate cancer cells.

In some embodiments, the method wherein the compound or composition specifically accumulates at prostate cancer cells.

In some embodiments, the method wherein detection of the compound or composition in the prostate gland of the subject is an indication that cancers cells are present in the prostate gland.

In some embodiments, the method wherein the cancer cells have elevated levels of prostate-specific membrane antigen (PSMA).

In some embodiments, the method wherein the compound or composition is detected using a PET imaging device.

The present invention provides a method of imaging prostate cancer cells in a subject comprising:
1) administering to the subject an effective amount of the metal complex of the present invention or a pharmaceutically acceptable salt thereof, or the composition of the present invention, wherein the compound specifically accumulates at prostate cancer cells in the subject;
3) detecting in the subject the location of the metal complex or the composition; and
4) obtaining an image of the cancer cells in the subject based on the location of the metal complex or the composition in the subject.

In some embodiments, the method wherein the image obtained is a three-dimensional image.

The present invention provides a method of detecting the presence of prostate cancer cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the prostate cancer cells based on the amount of the metal complex or composition determined to be present in the subject.

In some embodiments, the method wherein the detecting is performed by a Positron Emission Tomography (PET) device.

In some embodiments, the method further comprising quantifying the amount of the compound in the subject and comparing the quantity to a predetermined control.

In some embodiments, the method further comprising determining whether the subject is afflicted prostate cancer based on the amount of the compound in the subject.

In some embodiments, the method further comprising determining the stage of the prostate cancer.

The present invention provides a method of reducing the size of a prostate tumor or of inhibiting proliferation of prostate cancer cells comprising contacting the tumor or cancer cells with the metal complex of the present invention or a pharmaceutically acceptable salt thereof, or the composition of the present invention, so as to thereby reducing the size of the tumor or inhibit proliferation of the cancer cells.

In some embodiments, a compound having the structure:

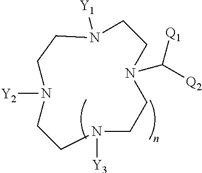

wherein
n is 0 or 1;
Y₁, Y₂ and Y₃ are each, independently, —H, alkylheteroaryl, alkyl-CO₂H, alkylaryl-CO₂H, alkylheteroaryl-CO₂H, alkyl-CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH) (alkyl-CO₂H), alkyl-N(alkylheteroaryl-OH) (alkyl-CO₂H), alkyl-P(O) (OH)₂, alkylaryl-P(O) (OH)₂ and alkylheteroaryl-P(O) (OH)₂,
   wherein each occurrence of R₄ is, independently, —H, alkyl, alkenyl, alkynyl, alkyl-aryl, alkyl-heteroaryl, aryl, heteroaryl, alkyl-CF₃, or —Si(alkyl)₃;
      wherein each occurrence of R₅ is, independently, —H, alkyl, alkenyl, alkynyl, alkyl-aryl, alkyl-heteroaryl, aryl, heteroaryl, alkyl-CF₃ or —Si (alkyl);
Q₁ is —CO₂H, —CO₂R₆, aryl-CO₂H, heteroaryl-CO₂H, aryl-CO₂R₆ or heteroaryl-CO₂R₆,
   wherein each occurrence of R₆ is, independently, —H, alkyl, alkenyl, alkynyl, alkyl-aryl, alkyl-heteroaryl, aryl, heteroaryl, alkyl-CF₃ or —Si(alkyl)₃; and
Q₂ is —H, CO₂H, alkyl-CO₂H, alkyl-CO₂(alkyl), alkyl-OH, alkyl-NH₂, alkyl-SH or alkyl-C(O)H,

or a salt of the compound.

In some embodiments of any of the disclosed compounds, Y₁ is —H and Y₂ is other than H.

In some embodiments of any of the disclosed compounds, Y₁ and Y₂ are each —H and Y₃ is other than H.

In some embodiments of any of the disclosed compounds, Y₁ and Y₃ are each —H and Y₂ is other than H.

In some embodiments of any of the disclosed compounds, Y₂ and Y₃ are each —H and Y₁ is other than H.

In some embodiments, a method for reducing one or more symptoms of disease in a subject, comprising administering an effective amount of the compound of the present invention or the composition of the present invention to the subject so as to treat the disease in the subject.

In some embodiments, the disease is cancer.

In some embodiments, the cancer cells have elevated levels of proteins or antigens or both.

In some embodiments, the compound wherein the metal is a radioisotope.

The present invention provides a pharmaceutical composition comprising a compound of the present invention and a pharmaceutically acceptable carrier.

The present invention provides a method for detecting cancer cells in a subject comprising administering an effective amount of a compound of the present invention or a composition of the present invention to the subject, and imaging the subject with a molecular imaging device to detect the compound or composition in the subject.

In some embodiments of the method, wherein the compound or composition specifically accumulates in cancer cells relative to non-cancer cells.

In some embodiments of the method, wherein detection of the compound or composition in an organ of the subject is an indication that cancers cells are present in the organ.

In some embodiments of the method, wherein the cancer cells are lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, or gall bladder cancer cells.

In some embodiments, the invention provides a method of reducing one or more symptoms of cancer or of imaging cancer cells. Cancers or cells thereof include, but are not limited to, lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

In some embodiments of the method, wherein the compound or composition is detected using a PET imaging device.

In some embodiments of the above method, the image obtained is a two-dimensional image.

In some embodiments of the above method, the image obtained is a three-dimensional image.

Methods of the present invention relate to the administration of a compound containing an imaging moiety linked to a targeting moiety, i.e. an antibody, peptide or small molecule, that recognizes target proteins or antigens in or on target cells in a subject.

The claimed conjugates are capable of high affinity binding to receptors on cancer cells or other cells to be visualized. The high affinity binding can be inherent to the targeting moiety or the binding affinity can be enhanced by the use of a derivative or fragment of the targeting moiety or by the use of particular chemical linkage between the imaging agent and targeting moiety that is present in the conjugate.

Imaging Agent

As used herein, the term “imaging agent” refers to any agent or portion (i.e. imaging moiety) of an agent that is used in medical imaging to visualize or enhance the visualization of the body including, but not limited to, internal organs, cells, cancer cells, cellular processes, tumors, and/or normal tissue. Imaging agents or imaging moieties include, but are not limited to, PET imaging agents. Imaging agents or moieties include, but are not limited to, any compositions useful for imaging cancer cells.

The imaging moiety of the compound of the present invention has the structure:

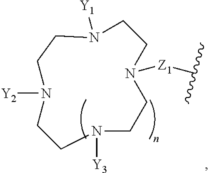

Targeting Agent

The targeting moiety may comprise, consist of, or consist essentially of an antibody, peptide or small molecule.

The targeting moiety may comprise, consist of, or consist essentially of Brentuximab (targets cell-membrane protein CD30), Inotuzumab targets CD22), Gemtuzumab (targets CD33), Milatuzumab (targets CD-74), Trastuzumab (targets HEP2 receptor), Glembatumomab (targets transmembrane glycoprotein NMB-GPNMMB), Lorvotuzumab (targets CD56), or Labestuzumab (targets carcinoembryonic cell adhesion molecule 5) or derivatives or fragments thereof.

The targeting moiety may comprise, consist of, or consist essentially of DUPA [(2-[3-(1, 3-dicarboxy propyl)ureido] pentanedioic acid)](targets prostate-specific membrane antigen (PSMA)), or derivatives or fragments thereof.

The targeting moiety may comprise, consist of, or consist essentially of bombesin (targets G-protein-coupled receptors BBR1, -2, and -3) or somatostatin (targets Somatostatin receptor subtypes 1-5), or derivatives or fragments thereof.

The targeting moiety is capable of selectively binding to the population of cells to be visualized due to preferential expression on the targeted cells of a receptor for the targeting moiety. The binding site for the targeting moiety can include receptors or other proteins that are uniquely expressed, overexpressed, or preferentially expressed by the population of cells to be visualized. A surface-presented protein uniquely expressed, overexpressed, or preferentially expressed by the cells to be visualized is a receptor not present or present at lower amounts on other cells providing a means for selective, rapid, and sensitive visualization of the cells targeted for diagnostic imaging using the conjugates of the present invention.

Exemplary targeting moieties are described in U.S. Pat. Nos. 10,005,820 B2, 9,801,951 B2 or U.S. Patent Application Publication No. 2015/0105540 A1 the contents of which are hereby incorporated by reference.

Chemical Linker

The term “chemical linker” refers to a chemical moiety or bond that covalently attaches two or more molecules, such as a targeting moiety and an imaging moiety. The linker may be a cleavable linkers, e.g. pH-sensitive (acid-labile) linker, disulfide linker, a peptide linker, a β-glucuronide linkers or a hydrazine linker. The linker may be a non-cleavable linker, e.g. thioether, maleimidocaproyl, maleimidomethyl cyclohexane-1carboxylate, alkyl, alkylamido or amide linker.

Covalent bonding of the imaging agent, chemical linker and targeting moiety can occur through the formation of amide, ester or imino bonds between acid, aldehyde, hydroxy, amino, or hydrazo groups. For example, a carboxylic acid on the targeting moiety can be activated using carbonyldiimidazole or standard carbodiimide coupling reagents such as 1-ethyl-3-(3-dimethylaminopropyl)-carbodiimide (EDC) and thereafter reacted with the other component of the conjugate, or with a linker, having at least one nucleophilic group, i.e. hydroxy, amino, hydrazo, or thiol, to form the vitamin-chelator conjugate coupled, with or without a linker, through ester, amide, or thioester bonds.

Linkage of a targeting moiety to the imaging moiety may be achieved by any means known to those in the art, such as genetic fusion, covalent chemical attachment, noncovalent attachment (e.g., adsorption) or a combination of such means. Selection of a method for linking a Targeting Moiety to an imaging moiety will vary depending, in part, on the chemical nature of the targeting moiety.

Linkage may be achieved by covalent attachment, using any of a variety of appropriate methods. For example, the targeting moiety and imaging moiety may be linked using bifunctional reagents (linkers) that are capable of reacting with both the targeting moiety and imaging moiety and forming a bridge between the two.

The term “non-covalent linker” is used in accordance with its ordinary meaning and refers to a divalent moiety which includes at least two molecules that are not covalently linked to each other but do interact with each other via a non-covalent bond (e.g. electrostatic interactions (e.g. ionic bond, hydrogen bond, halogen bond) or van der Waals interactions (e.g. dipole-dipole, dipole-induced dipole, London dispersion).

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (NH₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

A photocleavable linker (e.g., including or consisting of a o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally is a cleavable linker that following cleavage the two separated entities (e.g., fluorescent dye, bioconjugate reactive group) do not further react and form a new orthogonally cleavable linker.

Exemplary linkers are described in U.S. Patent Application No. 2012/0322741 A1, U.S. Patent Application No. 2018/0289828 A1 and U.S. Pat. No. 8,461,117 B2 the contents of which are hereby incorporated by reference.

Antibody

An “antibody” as used herein is defined broadly as a protein that characteristically immunoreacts with an epitope (antigenic determinant) of an antigen. As is known in the art, the basic structural unit of an antibody is composed of two identical heavy chains and two identical light chains, in which each heavy and light chain consists of amino terminal variable regions and carboxy terminal constant regions. The antibodies of the present invention include polyclonal antibodies, monoclonal antibodies (mAbs), chimeric antibodies, CDR-grafted antibodies, humanized antibodies, human antibodies, catalytic antibodies, multispecific antibodies, as well as fragments, regions or derivatives thereof provided by known techniques, including, for example, enzymatic cleavage, peptide synthesis or recombinant techniques.

As used herein, “monoclonal antibody” means an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495-97 (1975), or may be made by recombinant DNA methods (see, e.g., U.S. Pat. No. 4,816,567). The monoclonal antibodies may also be isolated from phage display libraries using the techniques described, for example, in Clackson et al., Nature 352:624-28 (1991) and Marks et al., J. Mol. Biol. 222(3):581-97 (1991).

The term “hybridoma” or “hybridoma cell line” refers to a cell line derived by cell fusion, or somatic cell hybridization, between a normal lymphocyte and an immortalized lymphocyte tumor line. In particular, B cell hybridomas are created by fusion of normal B cells of defined antigen specificity with a myeloma cell line, to yield immortal cell lines that produce monoclonal antibodies. In general, techniques for producing human B cell hybridomas, are well known in the art [Kozbor et al., Immunol. Today 4:72 (1983); Cole et al., in Monoclonal Antibodies and Cancer Therapy, Alan R. Liss, Inc. 77-96 (1985)].

The term “epitope” refers to a portion of a molecule (the antigen) that is capable of being bound by a binding agent, e.g., an antibody, at one or more of the binding agent's antigen binding regions. Epitopes usually consist of specific three-dimensional structural characteristics, as well as specific charge characteristics.

“Humanized antibodies” means antibodies that contain minimal sequence derived from non-human immunoglobulin sequences. For the most part, humanized antibodies are human immunoglobulins (recipient antibody) in which residues from a hyper variable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curro Opin. Struct. Biol. 2:593-96 (1992), each of which is incorporated herein by reference.

Also encompassed by the term “antibody” are xenogeneic or modified antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, the entire contents of which are incorporated herein by reference.

Those skilled in the art will be aware of how to produce antibody molecules of the present invention. For example, polyclonal antisera or monoclonal antibodies can be made using standard methods. To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. Hybridoma cells can be screened immunochemically for production of antibodies which are specifically reactive with the oligopeptide, and monoclonal antibodies isolated.

Target Cells

The term “target cells” refers to the cells that are involved in a pathology and so are preferred targets for imaging or therapeutic activity. Target cells can be, for example and without limitation, one or more of the cells of the following groups: primary or secondary tumor cells (the metastases), stromal cells of primary of secondary tumors, neoangiogenic endothelial cells of tumors or tumor metastases, macrophages, monocytes, polymorphonuclear leukocytes and lymphocytes, and polynuclear agents infiltrating the tumors and the tumor metastases. The term “targeting moiety” and “targeting agent” refer to an antibody, aptamer, peptide, small molecule or other substances that binds specifically to a target. A targeting moiety may be an antibody targeting moiety (e.g. antibodies or fragments thereof) or a non-antibody targeting moiety (e.g. aptamers, peptides, small molecules or other substances that bind specifically to a target).

The term “target tissue” refers to target cells (e.g., tumor cells) and cells in the environment of the target cells.

The term “cancer” refers to any of a number of diseases characterized by uncontrolled, abnormal proliferation of cells, the ability of affected cells to spread locally or through the bloodstream and lymphatic system to other parts of the body (e.g., metastasize), as well as any of a number of characteristic structural and/or molecular features. A “cancerous cell” or “cancer cell” is understood as a cell having specific structural properties, which can lack differentiation and be capable of invasion and metastasis. Examples of cancers are, breast, lung, brain, bone, liver, kidney, colon, and prostate cancer

Exemplary targets are described in Avicenna J Med Biotechnol. 2019 January-March; 11 (1): 3-23, Nature Reviews Drug Discovery Volume 16, pages 315-337 (2017), the contents of which are hereby incorporated by reference.

### Other Definitions

As used herein, the term “amino acid” refers to any natural or unnatural amino acid including its salt form, ester derivative, protected amine derivative and/or its isomeric forms. Amino Acids comprise, by way of non-limiting example: Agmatine, Alanine Beta-Alanine, Arginine, Asparagine, Aspartic Acid, Cysteine, Glutamine, Glutamic Acid, Glycine, Histidine, Isoleucine, Leucine, Lysine, Methionine, Phenylalanine, Phenyl Beta-Alanine, Proline, Serine, Threonine, Tryptophan, Tyrosine, and Valine. The amino acids may be L or D amino acids.

The terms “peptide”, “polypeptide”, peptidomimetic and “protein” are used to refer to a polymer of amino acid residues. The terms apply to amino acid polymers in which one or more amino acid residues is an artificial chemical mimetic of a corresponding naturally occurring amino acid, as well as to naturally occurring amino acid polymers. These terms also encompass the term “antibody”. “Peptide” is often used to refer to polymers of fewer amino acid residues than “polypeptides” or “proteins”. A protein can contain two or more polypeptides, which may be the same or different from one another.

As used herein, the term “oligopeptide” refers to a peptide comprising of between 2 and 20 amino acids and includes dipeptides, tripeptides, tetrapeptides, pentapeptides, etc.

An amino acid or oligopeptide may be covalently bonded to an amine of another molecule through an amide linkage, resulting in the loss of an “OH” from the amino acid or oligopeptide.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As sued herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′ R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules.

Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

The compounds of the present invention can be synthesized according to general Schemes. Variations on the following general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

While the invention has been shown and described with reference to certain embodiments of the present invention thereof, it will be understood by those skilled in the art that various changes in from and details may be made therein without departing from the spirit and scope of the present invention and equivalents thereof.

Experimental Details

Materials and Methods

All starting materials were purchased from Acros Organics, Alfa Aesar, Sigma Aldrich, or TCI America and used without further purification. NMR spectra (¹H, ¹³C, HSQC, HMBC) were collected on a 700 MHz Advance III Bruker instrument at 25° C. and processed using TopSpin 3.5p17. ⁴⁵Sc-NMR was recorded on a 700 MHz Advance III Bruker instrument at 25° C. Chemical shifts are reported as parts per million (ppm). Mass spectrometry: low-resolution electrospray ionization (ESI) mass spectrometry and high-resolution (ESI) mass spectrometry was carried out at the Stony Brook University Institute for Chemical Biology and Drug Discovery (ICB&DD) Mass Spectrometry Facility with an Agilent LC/MSD and Agilent LC-UV-TOF spectrometers respectively. UV-VIS spectra were collected with the NanoDrop ¹C instrument (AZY1706045). Spectra were recorded from 200 to 900 nm in a quartz cuvette with 1 cm path length. HPLC: Preparative HPLC was carried out using a Shimadzu HPLC-20AR equipped with a Binary Gradient, pump, UV-Vis detector, manual injector on a Phenomenex Luna C18 column (250 mm×21.2 mm, 100 Å, AXIA packed). Method A (preparative purification method): A=0.1?. TFA in water, B=0.1% TFA in MeCN. Gradient: 0-5 min: 95? A. 5-24 min: 5-95% B gradient. Method B (preparative purification method): A=10⁻² M ammonium formate in water, B=10% 10 mM ammonium formate in water, 90% MeCN. Gradient: 0-5 min: 95% A. 5-24 min: 5-95% B gradient. RadioHPLC analysis was carried out using a Shimadzu HPLC-20AR equipped with a binary gradient, pump, UV-Vis detector, autoinjector and Laura radiodetector on a Gemini-NX C18 column (100 mm-3 mm, 110 Å, AXIA packed). Method C: A=0.1% TFA in water, B=0.1% TFA in MeCN with a flow rate of 0.8 mL/min, UV detection at 260 and 280 nm. Benzyl tert-butyl 2-(methylsulfonyloxy)glutarate and tert-Butyl 6-(bromomethyl)-2-pyridinecarboxylate were synthesized according to previously published procedures.

tert-Butyl 6-[(1,4,7-triazonan-1-yl)methyl]-2-pyridinecarboxylate (1). 1,4,7-Triazocyclonane (0.0380 g, 0.295 mmol, 1 eq), tert-Butyl 6-(bromomethyl)-2-pyridinecarboxylate (0.0801. g, 0.295 mmol, 1 eq) was dissolved with K₂CO₃ (0.0409 g, 0.295 mmol, 1 eq) in acetonitrile (3.0 mL). The reaction mixture was stirred overnight at room temperature and subsequently filtered to remove solids. Solvent was removed in vacuo and (1) was purified using reverse-phase chromatography (Method A, product elutes at 35% B) and isolated as t yellowish oil (0.0309 g, 0.097 mmol, 33%). ¹H NMR (400 MHz, CDCl₃) δ 7.96 (d, J=7.58 Hz, 1H, H-5), 7.89 (t, J=7.73 Hz, 1H, H-6), 7.40 (d, J=7.67 Hz, 1H, H-7), 4.17 (s, 2H, H-9), 2.97-4.25 (m, 12H, H-10, H-11, H-12), 1.58 (s, 9H, 1-H). ¹³C NMR (100 MHz, CDCl₃) 163.53 (C-3), 158.26 (C-4), 147.94 (C-8), 139.18 (C-6), 125.88 (C-7), 124.11 (C-5), 83.93 (C-2), 57.55 (C-9), 50.40 (C-10), 46.39 (C-12), 45.76 (C-11), 27.73 (C-1). Calculated monoisotopic mass for 1 (C₁₂H₂₃N₄O₂): 320.22; found: m/z=321.2 [M+H]⁺.

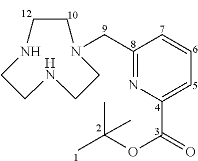

tert-Butyl 6-{[4-(tert-butoxycarbonylmethyl)-1,4,7-triazonan-1-yl]methyl}-2-pyridinecarboxylate (2) Compound 1 (0.517 g, 0.161 mmol, 1 eq), tert-Butyl bromoacetate (0.315. g, 0.161 mmol, 1 eq) was dissolved with K₂CO₃ (0.0224 g, 0.161 mmol, 1 eq) in acetonitrile (3.0 mL). The reaction mixture was stirred overnight at room temperature and subsequently filtered to remove solids. Solvent was removed in vacuo and (2) was purified using reverse-phase chromatography (Method A, product elutes at 35% B) and isolated as a yellowish oil (0.0457 g, 0.101 mmol, 65%). ¹H NMR (500 MHz, CDCl₃) δ 7.96 (d, J=7.25 Hz, 1H, H-7), 7.89 (t, J=7.80 Hz, 1H, H-6), 7.44 (d, J=7.67 Hz, 1H, H-5), 4.26 (s, 2H, H-16), 3.84 (s, 2H, H-9) 3.13-3.83 (m, 12H, H-10, H-11, H-12, H-14, H-15), 1.61 (s, 9H, H-1) 1.38 (s, 9H, H-19. ¹³C NMR (175 MHz, CDCl₁) 163.98 (C-17), 157.64 (C-3) 161.41 (C-4), 147.71 (C-8), 138.84 (C-6), 126.06 (C-7), 124.11 (C-5), 83.90 (C-18), 83.68 (C-2), 58.64 (C-9), 58.48 (C-16), 55.00 (C-15), 53.50 (C-12) 52.48 (C-10), 50.82 (C-11), 45.26 (C-14), 45.13 (C-13), 27.89 (C-19), 27.83 (C-1). Calculated monoisotopic mass for 2 (C₂₃H₃₈N₄O₄): 434.29; found: m/z=435.3 [M+H]⁺.

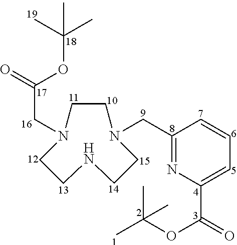

tert-Butyl 6-{[4,7-bis(tert-butoxycarbonylmethyl)-1,4,7-triazonan-1-yl]methyl}-2-pyridinecarboxylate (4a). Compound 2 (0.0517 g, 0.161 mmol, 1 eq) tert-Butyl bromoacetate (0.0315. g, 0.161 mmol, 1 eq) was dissolved with K₂CO₃ (0.0224 g, 0.161 mmol, 1 eq) in acetonitrile (3.0 mL). The reaction mixture was stirred overnight at room temperature and subsequently filtered to remove solids. Solvent was removed in vacuo and (2) was purified using reverse-phase chromatography (Method B, product elutes at % B) and isolated as a yellowish oil (0.0068 g, 0.0129 mmol, 8). ¹H NMR (500 MHz, CDCl₃) δ 8.14 (d, J=7.25 Hz, 1H, H-7), 7.92 (t, J=7.80 Hz, 1H, H-6), 7.47 (d, J=7.67 Hz, 1H, H-5), 4.88 (s, 2H, H-9), 4.22 (s, 4H, H-16), 3.09-3.68 (m, 12H, H-10, H-11, H-12, H-14, H-15), 1.53 (s, 18H, H-19), 1.43 (s, 9H, H-1). ¹³C NMR (175 MHz, CDCl₃) 168.85 (C-17), 163.61 (C-3) 160.95 (C-4), 148.81 (C-8), 138.57 (C-6), 126.00 (C-7), 124.77 (C-5), 83.03 (C-18), 82.79 (C-2), 59.41 (C-9), 56.98 (C-16), 52.09 (C-15, C-10, C-11, C-14), 49.88 (C-12, C-13), 28.00 (C-1, C-19). Calculated monoisotopic mass for 4a (C₂₉H₄₈N₄O₆): 548.36; found: m/z=549.5 [M+H]⁺.

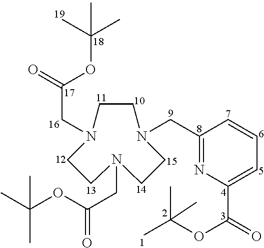

6-{[4,7-Bis(carboxymethyl)-1,4,7-triazonan-1-yl]methyl}-2-pyridinecarboxylic acid (7a). Compound 4a (0.0112 g, 0.020 mmol, 1 eq) was dissolved into as solution of 2:1 TFA and DCM (1 mL). The reaction mixture was stirred overnight at room temperature. Solvent was removed in vacuo and (7a) was purified using reverse-phase chromatography (Method B, product elutes at % B) and isolated as an off-white solid (0.0067 g, 0.018 mmol, 88%). ¹H NMR (400 MHz, CDCl₃): δ 8.17 (m, J=7.95 Hz, 1H, H-3), 8.04 (m, J=7.82 Hz, 1H, H-4), 7.82 (m, J=7.54 Hz, 1H, H-5), 4.45 (s, 2H, H-7), 3.68 (s, 4H, H-11), 3.01-3.27 (m, 12H, H-8, H-9, H-10). ¹³C NMR (100 MHz, CDCl₃): 171.99 (C-12), 166.04 (C-1), 154.74 (C-2), 147.87 (C-6), 138.67 (C-4), 127.39 (C-3), 124.51 (C-5), 59.01 (C-8), 54.69 (C-10), 50.62 (C-9), 49.96 (C-11), 48.81 (C-7). Calculated monoisotopic mass for 7a

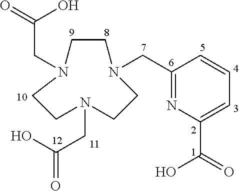 [SMILES: O=C(O)CN1CCN(CC(=O)O)CCN(Cc2cccc(C(=O)O)n2)CC1]

Benzyl tert-butyl 2-{7-(tert-butoxycarbonylmethyl)-4-[(6-tert-butoxycarbonyl-2-pyridyl)methyl]-1,4,7-triazonan-1-yl}glutarate (3b). Compound 2 (0.0440 g, 0.101 mmol, 1 eq), Benzyl tert-butyl 2-(methylsulfonyloxy)glutarate (0.0376 g, 0.101 mmol, 1 eq) was dissolved with K₂CO₃ (0.0140 g, 0.101 mmol, 1 eq) in acetonitrile (3.0 mL). The reaction mixture was stirred overnight at room temperature and subsequently filtered to remove solids. Solvent was removed in vacuo and (4b) was purified using reverse-phase chromatography (Method A, product elutes at 95% B) and isolated as a yellowish oil (0.0112 g, 0.016 mmol, 16%). NMR (400 MHz, CDCl₃): δ 7.98 (m, 1H, H-5), 7.87 (m, 1H, H-6), 7.65 (m, 1H, H-7), 7.33 (m, 5H, H-29, H-30, H-31), 5.05 (s, 2H, H-27), 4.61 (m, 2H, H-9), 2.63-3.67 (m, 15H, H-10, H-11, H-12, H-13, H-14, H-15, H-16, H-20), 2.52 (m, 2H, H-25), 2.01 (m, 2H, H-24), 1.37-1.70 (m, 27H, H-1, H-19, H-23). ¹³C NMR (175 MHz, CDCl₃): 174.52 (C-26), 172.76 (C-21), 171.23 (C-17), 169.30 (C-3), 160.56 (C-4), 160.24 (C-8), 148.95 (C-6), 138.78 (C-5), 135.69 (C-31), 128.59 (C-29), 128.43 (C-28), 128.27 (C-31), 127.09 (C-7), 82.65 (C-27), 66.47 (C-9), 64.17 (C-16), 59.10 (C-20), 28.10 (C-1), 28.01 (C-19), 28.00 (C-23), 27.87 (C-24), 25.02 (C-25). Calculated monoisotopic mass for 4b (C₃₉H₅₈N₄O₈): 710.43; found: m/z=711.4 [M+H]⁺.

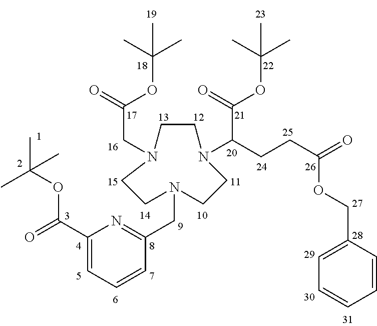 [SMILES: CC(C)(C)OC(=O)CN1CCN(Cc2cccc(C(=O)OC(C)(C)C)n2)CCN(C(CCC(=O)OCc2ccccc2)C(=O)OC(C)(C)C)CC1]

4-tert-Butoxycarbonyl-4-(7-(tert-butoxycarbonylmethyl)-4-[(6-tert-butoxycarbonyl-2-pyridyl)methyl]-1,4,7-1 m triazonan-1-yl)butyric acid (4). Compound 3b (0.0312 g, 0.044 mmol, 1 eq) was dissolved in EtOH (4 mL) and 10% Pd/C (0.0120 g) was added to the flask. After purging the flask with H₂, the reaction mixture was stirred for 3 h under light H₂-pressure (balloon). The reaction mixture was filtered through a PVDF filter, the solvent was evaporated in vacuo, and the desired product was obtained as a yellow oil (0.0272 g, 0.044 mmol, 99%) and used without further purification immediately for amidation with protected DUPA fragment. Calculated monoisotopic mass for 4 (C₃₂H₅₂N₄O₈): 620.38; found: m/z=621.3 [M+H]⁺.

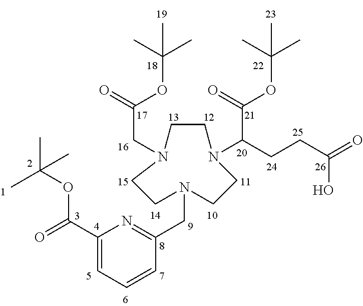 [SMILES: CC(C)(C)OC(=O)CN1CCN(Cc2cccc(C(=O)OC(C)(C)C)n2)CCN(C(CCC(=O)O)C(=O)OC(C)(C)C)CC1]

### Ditert-butyl 2-{3-[(R)-4-oxo-1-tert-butoxycarbonyl-4-[5-(4-tert-butoxycarbonyl-4-{7-(tert-butoxycarbonylmethyl)-4-[(6-tert-butoxycarbonyl-2-pyridyl)methyl]-1,4,7-triazonan-1-yl)butyrylamino)pentylamino]butyl]ureido}glutarate (5)

Compound 4 (0.0272, 0.0439, 1.0 eq) and HBTU (0.0183 g, 0.0439, 1.1 eq) were dissolved in DMF (1 mL), DIPEA (0.0057 g, 0.0439 mmol, 1.1 eq) was added. Ditert-butyl 2-(3-[(R)-4-(5-aminopentylamino)-4-oxo-1-tert-butoxycarbonylbutyl]ureido}glutarate (C) (0.0251 g, 0.0483 mmol, 1 eq) was added and reaction mixture was stirred overnight at room temperature. Solvent was removed in vacuo and product solution was purified by reverse-phase flash chromatography to afford the title compound (0.0045 g, 0.004 mmol, 9?) as a colorless solid. NMR (500 MHz, CDCl₃): δ 8.00 (d, 1H, H-5), 7.91 (t, 1H, H-6), 7.66 (d, 1H, H-7), 4.18 (m, 2H, H-35, H-37), 3.56 (m, 2H, H-16), 3.38 (m, 1H, H-20), 3.12 (m, 4H, H-27, H-31), 2.70-3.69 (m, 14H, H-9, H-10, H-11, H-12, H-13, H-14, H-15), 2.35 (m, 2H, H-25), 2.28 (m, 2H, H-42), 2.20 (m, 2H, H-33), 2.03 (m, 2H, H-34, H-41), 2.02 (m, 1H, H-24), 1.91 (m, 1H, H-24), 1.79 (m, 2H, H-34, H-41), 1.43 (m, 4H, H-28, H-30), 1.33-1.59 (m, 63H, H-1, H-19, H-23, H-40, H-45, H-48) 1.28 (m, 2H, H-29). ¹³C NMR (100 MHz, CDCl₃): 173.26 (C-26), 173.41, (C-32), 173.33 (C-38), 172.36 (C-46), 172.33 (C-43), 172.10 (C-21), 171.97 (C-17), 171.78 (C-36), 169.98 (C-3), 148.94 (C-4), 138.86 (C-8), 124.83 (C-6), 116.76 (C-5), 115.08 (C-7), 82.73 (C-22), 82.52 (C-47), 81.76 (C-44), 81.70 (C-18), 81.46 (C-2), 81.43 (C-39), 64.12 (C-20), 58.74 (C-35), 58.21 (C-37), 53.19 (C-19), 52.78 (C-16), 31.07 (C-25), 28.72 (C-33), 28.64 (C-34), 28.46 (C-24), 27.89 (C-29), 27.09 (C-23), 27.05 (C-45), 27.01 (C-48), 26.96 (C-19), 26.90 (C-1) 25.69 (C-41), 25.61 (C-42), 23.87 (C-40). Calculated monoisotopic mass for 5 (C₆₀H₁₀₂N₈O₁₅): 1174.75; found: m/z=1175.8 [M+H]⁺.

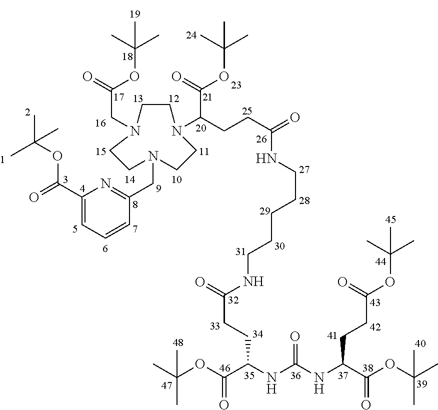 [SMILES: CC(C)(C)OC(=O)CC[C@H](NC(=O)N[C@@H](CCC(=O)NCCCCCNC(=O)CCC(C(=O)OC(C)(C)C)N1CCN(CC(=O)OC(C)(C)C)CCN(Cc2cccc(C(=O)OC(C)(C)C)n2)CC1)C(=O)OC(C)(C)C)C(=O)OC(C)(C)C]

### 2-{3-[(R)-1-Carboxy-4-[5-(4-carboxy-4-{7-(carboxymethyl)-4-[(6-carboxy-2-pyridyl)methyl]-1,4,7-triazonan-1-yl}butyrylamino)pentylamino]-4-oxobutyl]ureido}glutaric acid (6)

Compound 5 (0.0045 g, 0.004 mmol, 1 eq) was dissolved into as solution of 2:1 TFA and DCM (1 mL). The reaction mixture was stirred overnight at room temperature. Solvent was removed in vacuo and 6 was purified using reverse-phase chromatography (Method B, product elutes at 15% B) and isolated as an off-white solid (0.0034 g, 0.004 mmol, 99%). NMR (700 MHz, CDCl₃): δ 8.19 (d, 1H, H-5), 8.07 (m, 1H, H-6), 7.84 (m, 1H, H-7), 4.33 (m, 1H, H-35), 4.29 (m, 1H, H-37), 3.70 (m, 2H, H-16), 3.47 (m, 1H, H-20), 3.15 (m, 4H, H-27, H-31), 2.89-3.60 (m, 14H, H-9, H-10, H-11, H-12, H-13, H-14, H-15), 2.43 (m, 2H, H-25), 2.31 (m, 2H, H-42), 2.18 (m, 2H, H-33), 2.02 (m, 1H, H-24), 1.63 (m, 1H, H-24), 1.90 (m, 2H, H-34, H-41), 1.51 (m, 2H, H-34, H-41), 1.31 (m, 4H, H-28, H-30), 0.92 (m, 2H, H-29). ¹³C NMR (175 MHz, CDCl₃): 175.03 (C-26), 174.53, (C-32), 174.40 (C-38), 173.56 (C-46), 173.50 (C-43), 166.00 (C-21), 160.48 (C-17), 160.28 (C-36), 158.71 (C-3), 131.17 (C-4), 131.02 (C-8), 126.83 (C-6), 117.00 (C-5), 115.35 (C-7), 67.73 (C-20), 58.80 (C-16), 52.20 (C-35), 52.18 (C-37), 38.85 (C-25), 31.89 (C-33), 29.68 (C-34), 28.58 (C-24), 28.50 (C-29), 27.39 (C-23), 25.28 (C-41), 23.80 (C-42). Calculated monoisotopic mass for 6 (C₂₆H₅₄N₈O₁₅): 838.37; found: m/z=839.1 [M+H]⁺.

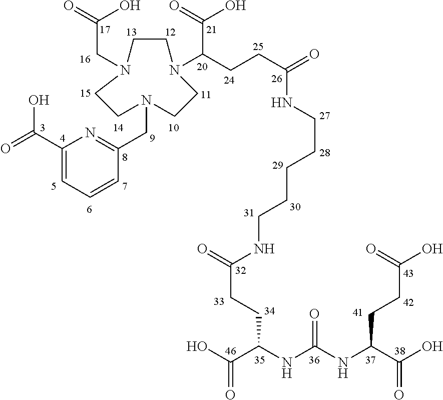 [SMILES: O=C(O)CC[C@H](NC(=O)N[C@@H](CCC(=O)NCCCCCNC(=O)CCC(C(=O)O)N1CCN(CC(=O)O)CCN(Cc2cccc(C(=O)O)n2)CC1)C(=O)O)C(=O)O]

### Benzyl tert-butyl 2-(1,4,7-triazonan-1-yl)glutarate (11)

1,4,7-Triazocyclonane (0.703 g, 0.546 mmol, 1 eq), Benzyl tert-butyl 2-(methylsulfonyloxy)glutarate (0.2030 g, 0.546 mmol, 1 eq) was dissolved with K₂CO₃ (0.0759 g, 0.546 mmol, 1 eq) in acetonitrile (3.0 mL). The reaction mixture was stirred overnight at room temperature and subsequently filtered to remove solids. Solvent was removed in vacuo and (11) was purified using reverse-phase chromatography and isolated as a white solid (0.1397 g, 0.346 mmol, 63). ¹H NMR (500 MHz, CDCl₃) δ 7.36 (b, 5H, H-1, H-2, H-3), 5.13 (m, 2H, H-5), 3.39 (1H, H-9) 2.92-3.85 (m, 12H, H-13, H-14, H-15), 2.48 (m, 2H, H-7), 2.57 (m, 2H, H-7), 2.04 (m, 2H, H-8), 1.45 (s, 9H, H-12). ¹³C NMR (175 MHz, CDCl₃) 172.68 (C-6), 172.51 (C-10), 135.66 (C-1), 128.64 (C-4), 128.44 (C-3), 128.35 (C-2), 83.81 (C-11), 66.63 (C-9), 45.54 (C-13, C-14, C15), 31.32 (C-8), 28.00 (C-12), 24.38 (C-7). Calculated monoisotopic mass for 11 (C₂₂H₃₅N₃O₄): 405.26; found m/z=406.2 [M+H]⁺.

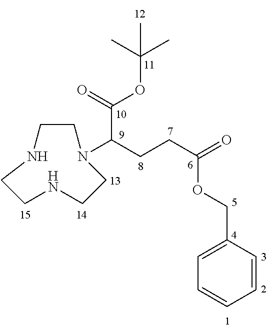 [SMILES: CC(C)(C)OC(=O)C(CCC(=O)OCc1ccccc1)N1CCNCCNCC1]

### Benzyl tert-butyl 2-[4-(tert-butoxycarbonylmethyl)-1,4,7-triazonan-1-yl]glutarate (11)

Compound 12 (0.1397 g, 0.346 mmol, 1 eq), tert-Butyl bromoacetate (0.386 g, 0.193 mmol, 0.6 eq) was dissolved with KₖCO₃ (0.0477 g, 0.3458 mmol, 1 eq) in acetonitrile (3.0 mL). The reaction mixture was stirred overnight at room temperature and subsequently filtered to remove solids. Solvent was removed in vacuo and (110 was purified using reverse-phase chromatography and isolated as a yellowish oil (0.0444 g, 43%). ¹H NMR (400 MHz, CDCl₃) δ 7.37 (b, 5H, H-1, H-2, H-3), 5.14 (s, 2H, H-5), 3.39 (s, 2H, H-16), 2.65-3.29 (m, 13H, H-9, H-13, H-14, H-15), 2.48 (m, 2H, H-7), 2.12 (m, 1H, H-8), 1.93 (m, 1H, H-8), 1.47 (s, 9H, H-12, 1.46 (s, 9H, H-19). ¹³C NMR (100 MHz, CDCl₃) 172.68 (C-6), 171.5 (C-10), 170.68 (C-20), 135.78 (C-1), 128.60 (C-4), 128.41 (C-3), 128.39 (C-2), 82.42 (C-Il), 82.35 (C-21), 66.56 (C-19), 64.00 (C-9), 56.31 (C-16) 48.35 (C-17), 47.66 (C-13), 46.12 (C-18), 44.70 (C-15), 44.26 (C-14), 30.80 (C-8), 28.10 (C-12), 28.00 (C-22), 24.78 (C-7). Calculated monoisotopic mass for 4 (C₂₈H₄₅N₃O₄): 519.33; found: m/z=520.4 [M+H]⁺.

[symbol]

### Benzyl tert-butyl 2-{4-[(6-tert-butoxycarbonyl-2-pyridyl)methyl]-1,4,7-triazonan-1-yl}glutarate (12)

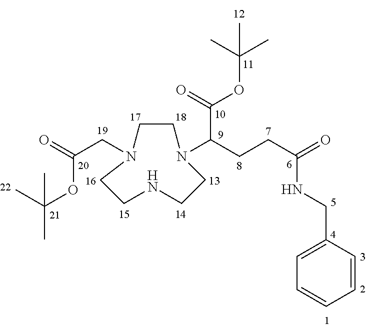 [SMILES: CC(C)(C)OC(=O)CN1CCNCCN(C(CCC(=O)NCc2ccccc2)C(=O)OC(C)(C)C)CC1]

Compound 11 (0.271 g, 0.067 mmol, 1 eq), tert-Butyl 6-(bromomethyl)-2-pyridinecarboxylate (0.0182 g, 0.067 mmol, 1 eq) was dissolved with K₇CO₃ (0.0093 g, 0.067 mmol, 1 eq) in acetonitrile (3.0 mL). The reaction mixture was stirred overnight at room temperature and subsequently filtered to remove solids. Solvent was removed in vacuo and (1) was purified using reverse-phase chromatography (Method A, product elutes at 35% B) and isolated as a yellowish oil (0.0078 g, 0.013 mmol, 20). ¹H NMR (500 MHz, CDCl₃) δ 7.89 (m, 1H, H-5), 7.81 (m, 1H, H-6), 7.36 (m, 6H, H-7, H-25, H-26, H-27), 5.09 (s, 2H, H-23), 4.16 (m, 2H, H-9), 2.78-3.63 (m, 13H, H-10-H-16), 2.49 (m, 2H, H-21), 2.07 (m, 1H, H-20), 1.91 (m, 1H, H-20), 1.59 (m, 9H, H-19), 1.43 (s, 9H, H-1)³C NMR. (100 MHz, CDCl₃) 172.59 (C-22), 171.45 (C-17), 163.56 (C-3), 158.21 (C-4), 157.28 (C-8), 148.15 (C-6), 138.39 (C-5), 135.69 (C-27), 128.64 (C-25), 128.40 (C-26), 125.62 (C-24), 83.41 (C-2), 82.37 (C-18), 66.56 (C-23), 65.27 (C-9), 58.77 (C-15) 50.89 (C-14), 49.63 (C-10, C-12), 47.75 (C-11), 45.59 (C-13, C-14), 39.90 (C-16), 32.80 (C-20), 31.07 (C-21), 28.11 (C-19), 27.88 (C-1). Calculated monoisotopic mass for 12 (C₃₃H₄₈N₄O₆): 596.36; found: m/z=597.3 [M+H]⁺.

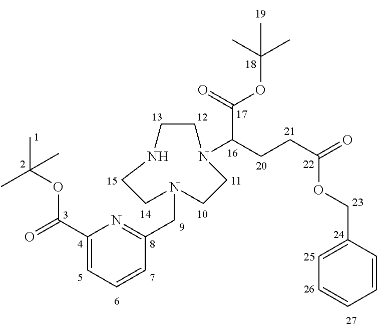 [SMILES: CC(C)(C)OC(=O)c1cccc(CN2CCNCCN(C(CCC(=O)OCc3ccccc3)C(=O)OC(C)(C)C)CC2)n1]

### (4-{2-[2-(5-((R)-4-[3-(1,3-Ditert-butoxycarbonylpropyl)ureido]-4-tert-butoxycarbonylbutyrylamino)pentylamino)-2-oxoethylamino]-2-oxoethyl}-7,10-bis(carboxymethyl)-1,4,7,10-tetraaza-1-cyclododecyl)acetic acid (8)

Ditert-butyl 2-{3-[(R)-4-(5-aminopentylamino)-4-oxo-1-tert-butoxycarbonylbutyl]ureido}glutarate (C) (0.0100 g, 0.0175 mmol, 1 eq) was added to {4,10-Bis(carboxymethyl)-7-[(2,5-dioxo-1-pyrrolidinyloxycarbonyl)methyl]-1,4,7,10-tetraaza-1-cyclododecyl}acetic acid.HPF₆.TFA (13.8 g, 0.0175 mmol, 1 eq) and DIPEA (0.0023 g, 0.0175 mmol, 1 eq) in 1 mL DMF. The reaction mixture was stirred for 2 h at room temperature. Mixture was concentrated in vacuo and 8 was purified using reverse-phase chromatography (Method B, product elutes at 60% B) and isolated as an off-white solid (0.0079 g, 0.008 mmol, 47%). NMR (700 MHz, MeOD): δ 4.22 (m, 1H, H-11), 4.15 (m, 1H, H-9), 3.27-3.38 (m, 22H, H-19, H-20, H-21, H-22, H-23, H-25), 3.12-3.26 (m, 6H, H-1, H-5, H-18), 2.31 (m, 4H, H-7, H-15), 2.09 (m, 2H, H-8, H-14), 1.84 (m, 2H, H-8, H-14), 1.54 (m, 4H, H-2, H-4), 1.49 (m, 18H, H-28, H-30), 1.47 (m, 9H, H-30), 1.37 (m, 2H, H-3). ¹³C NMR (100 MHz, MeOD) 172.34 (C-6), 172.12 (C-12, C-13), 172.02 (C-16), 159.71 (C-17), 159.47 (C-24), 159.27 (C-26), 158.44 (C-10), 116.59 (C-23), 114.96 (C-25), 81.53 (C-27), 81.49 (C-31), 80.40 (C-29), 53.21 (C-9), 52.81 (C-18), 48.15 (C-11), 47.96 (H-19, H-20, H-21, H-22), 39.04 (C-1), 38.81 (C-5), 31.81 (C-2), 31.78 (C-4), 31.07 (C-15), 28.69 (C-8), 28.37 (C-14), 27.58 (C-7), 26.95 (C-28, C-32), 26.89 (C-30), 23.76 (C-3). Calculated monoisotopic mass for 8 (C₄₄H₇₈N₈O₁₅): 958.56; found: m/z=959.5 [M+H]⁺.

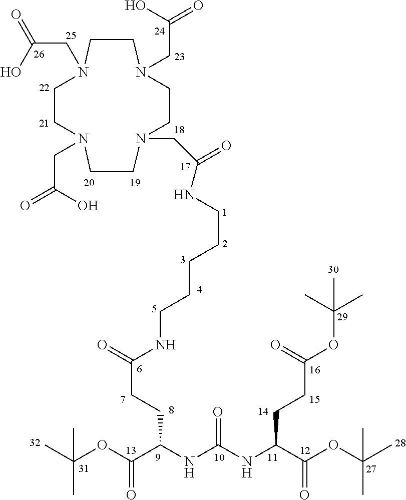 [SMILES: CC(C)(C)OC(=O)CC[C@H](NC(=O)N[C@@H](CCC(=O)NCCCCCNC(=O)CN1CCN(CC(=O)O)CCN(CC(=O)O)CCN(CC(=O)O)CC1)C(=O)OC(C)(C)C)C(=O)OC(C)(C)C]

### 2-{3-[(R)-1-Carboxy-4-oxo-4-[5-(2-{2-[4,7,10-tris(carboxymethyl)-1,4,7,10-tetraaza-1-cyclododecyl]acetylamino}acetylamino) pentylamino]butyl]ureido}glutaric acid (9)

Compound 8 (0.0138 g, 0.167 mmol, 1 eq) was dissolved into as solution of 2:1 TFA and DCM (1 mL). The reaction mixture was stirred overnight at room temperature. Solvent was removed in vacuo and 9 was purified using reverse-phase chromatography (Method B, product elutes at 15% B) and isolated as an off-white solid (0.0058 g, 0.007 mmol, 53%). NMR (400 MHz, D₂O): 4.20 (m, 1H, H-9), 4.10 (m, 1H, H-11), 3.54-4.00 (b, 8H, H-18, H-23, H-25, H-22, H-23, H-25), 2.95-3.45 (m, 20H, H-1, H-5, H-19, H-20, H-21, H-22), 2.54 (m, 2H, H-15) 2.28 (m, 2H, H-7), 2.10 (m, 2H, H-8, H-14), 1.90 (m, 2H, H-8, H-14), 1.43 (m, 4H, H-2, H-4), 1.24 (m, 2H, H-3). ¹³C NMR (100 MHz, MeOD) 175.03 (C-6), 174.61 (C-12, C-13), 174.48 (C-16), 173.54 (C-17), 161.42 (C-26), 161.15 (C-24), 158.74 (C-10), 81.26 (C-23), 78.13 (C-25), 52.23 (C-9), 52.21 (C-11), 49.32 (C-18), 49.32 (H-19), 48.46 (H-20), 48.13 (H-21), 48.07 (H-22), 38.64 (C-1), 33.94 (C-5), 33.79 (C-2), 31.77 (C-4), 29.66 (C-15), 28.63 (C-8), 28.36 (C-14), 27.36 (C-7), 23.70 (C-3). Calculated monoisotopic mass for 9 (C₃₂H₅₄N₈O₁₅): 790.37; found: m/z=791.3 [M+H]⁺.

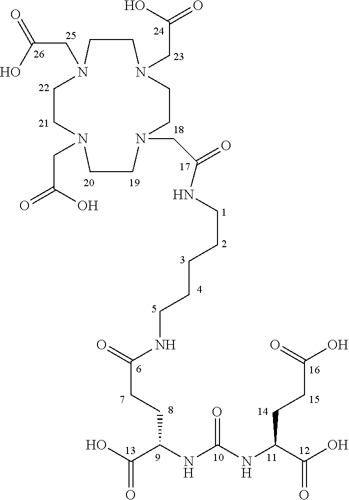 [SMILES: O=C(O)CC[C@H](NC(=O)N[C@@H](CCC(=O)NCCCCCNC(=O)CN1CCN(CC(=O)O)CCN(CC(=O)O)CCN(CC(=O)O)CC1)C(=O)O)C(=O)O]

Nonradioactive Scandium Complexes

To obtain a single Sc-species in solution, the following general protocol was employed: Ligand (0.02 mmol), previously deprotected under acidic conditions was dissolved in DI H₂O (1 mL). Solutions of ScCl₃.6H₂O (0.02 mmol) or LuCl₃.6H₂O (0.01 mmol) were each dissolved in H₂O (1 mL) and one-half molar equivalent added to each ligand solution. The pH of the resulting acidic solution was subsequently adjusted from pH 3 to 6 by drop-wise addition of NaOH (1 M solution in H₂O). The mixture was subsequently heated at 80° C. for 0.5 hours to ensure complete complexation. The resulting aqueous solutions were lyophilized overnight to afford the lanthanide or scandium complex as an off-white powder.

Na[Sc(DOTA)]: ¹H NMR (400 MHz, D₂O): ⁴⁵Sc NMR (400 MHz, D₂O): 90.9 ppm Calculated monoisotopic mass for (C₁₆H₂₅N₄O₈Sc): 446.12; found: m/z=447.1 [M+H]⁺.

Na[Lu (DOTA)]: Calculated monoisotopic mass for (C₁₆H₂₅N₄O₈Lu): 576.11; found: m/z=577.0 [M+H]⁺.

[Sc(7a)]: ¹H NMR (400 MHz, D₂O): 8.13 (t, J=7.85 Hz, 1H, H-3), 7.97 (d, J=7.45 Hz, 1H, H-4), 7.61 (d, J=7.85 Hz, 1H, H-5), 4.44 (s, 2H, H-7), 3.78 (m, 2H, H-11), 3.43 (m, 2H, H-11), 2.93-3.24 (m, 12H, H-8, H-9, H-10). ₄₅Sc NMR (400 MHz, D₂O): 78.8 ppm Calculated monoisotopic mass for (C₁₇H₂₁N₄O₆Sc): 422.10; found: m/z=423.1 [M+H]⁺.

[Lu(7a)]: ¹H NMR (400 MHz, D₂O): δ 8.16 (t, J=7.73 Hz, 1H, H-3), 8.02 (d, J=7.73 Hz, 1H, H-4), 7.82 (d, J=7.91 Hz, 1H, H-5), 4.43 (s, 2H, H-7), 3.74 (m, 2H, H-11), 3.45 (m, 2H, H-11), 2.90-3.23 (m, 12H, H-8, H-9, H-10). Calculated monoisotopic mass for (C₁₇H₂₁N₄O₆Lu): 552.09; found: m/z=553.0 [M+H]⁺.

[Sc(7a)]: ¹H NMR (400 MHz, D₂O): ⁴⁵Sc NMR (400 MHz, D₂O): 50.5 ppm Calculated monoisotopic mass for (C₁₇H₂₁N₄O₆Sc): 422.10; found: m/z=423.1 [M+H]⁺.

[Lu(7a)]: ¹H NMR (400 MHz, D₂O): Calculated monoisotopic mass for (C₁₇H₂₁N₄O₆Lu): 552.09; found: m/z=553.0 [M+H]⁺.

Imaging and Biodistribution

All animal experiments were conducted according to the guidelines of the Institutional Animal Care and Use Committee (IACUC) at Stony Brook Medicine. Male NCr nude mice (6 weeks, Taconic Biosciences, Rensselaer, N.Y.) were implanted subcutaneously on the right shoulder with 0.7-0.9×10⁶ PC3-PIP cells and on the left shoulder with 0.7-0.9×10⁶ PC-3 flu cells suspended in Matrigel (1:1). When the tumors reached 50-100 mm³, the mice were anesthetized with isoflurane, and 0.6-3.0 MBq (15-82 μCi) of the tracer (3-30 μg) was intravenously injected via tail vein catheter. Mice were imaged at 30, 60, and 90 min post injection (p.i.) using Siemens Inveon PET/CT Multimodality System, and images were reconstructed using ASIPro software. Region of interest (ROI) analyses on all images were performed using AMIDE. Upon completion of imaging at 120 min p.i., mice were sacrificed, and select organs were harvested. Radioactivity was counted by using a gamma counter, and the radioactivity associated with each organ was expressed as % ID/g. Biodistribution data were assessed by unpaired t-tests using GraphPad Prism to determine if differences between groups were statistically significant (p<0.05).

Radioactive Scandium Complexes

General Methods. Method E: RadioHPLC. UV absorption was recorded at 254 and 280 ran, samples were analyzed using a C18 column (Acclaim 120, 250 mm×4.60 mm), 1 mL/min flow, with mobile phase method 1: Solvent A: water, solvent B=acetonitrile. 100000 ng/mL complexed at 80° C. were used for HPLC characterization. TLC plates were developed in 0.1 M sodium citrate and read on Packard Cyclone Phosphor scanner. Plates were quantitated on OptiQuant software.

Concentration- and temperature dependent radiolabeling experiments at 25 and 80° C. A stock solution of ligand was diluted to produce a 10⁵ ng/mL solution in DI water. 10⁴, 3×10³, 10³, 10² ng/mL, and 10 ng/mL concentrations were prepared from the original solution by serial dilution. 0.1 mL of each ligand solution was mixed with sodium acetate (0.1 mL 0.25 M, pH 4.5). ⁴⁴Sc was eluted in dilute HCl (0.1M ?). 100 uCi of activity was added to each tube and the reaction was vortexed for 20 seconds. Subsequently, tubes were either placed in a heating block at 80° C. or were kept at room temperature. Tubes were intermittently vortexed and 2 uL was removed at the designated timepoints and spotted onto TLC plates or analyzed using Method E.

Challenge experiments. To measure relative complex stability, 10 uL of ligand solution at 100000 ng/mL was added to 100 uL EDTA to produce 10× excess EDTA. Mixtures were incubated at 37° C. for 1 h. To evaluate plasma stability, 10 uL of ligand solution at 100000 ng/mL added to 50 uL mouse plasma. Incubated at 37° C. for 1 h. Tubes were intermittently vortexed and 2 uL was removed at the designated timepoints and spotted onto TLC plates.

TABLE 1
Challenge Experiments

| | Ligand [time point, % complex stability] | | | |
|---|---|---|---|---|
| Challenge Experiment | t = 0 | t = 10 min | t = 30 min | t = 60 min |
| | DOTA | | | |
| EDTA, 10× eq, 37° C. | 9.41 | 74.4 ± 0.4 | 73.3 ± 0.5 | 74.7 ± 0.4 |
| Rat Plasma, 37° | | 74.8 ± 1.8 | 74.4 ± 0.7 | 75.5 ± 0.9 |
| | MONOPIC | | | |
| EDTA, 10× eq, 37° C. | 91.4 | 91.5 ± 0.8 | 91.4 ± 0.8 | 90.5 ± 0.6 |
| Rat Plasma, 37° | | 90.1 ± 0.8 | 91.0 ± 0.2 | 91.6 ± 0.3 |
| | NO2PIC | | | |
| EDTA, 10× eq, 37° C. | 90.2 | 77.4 ± 5.2 | 67.8 ± 0.9 | 51.5 ± 0.4 |
| Rat Plasma, 37° | | 98.9 ± 0.3 | 98.3 ± 0.1 | 97.4 ± 0.4 |

TABLE 2
Molar Acitvity
Apparent Molar Activity (Ci/μmol)

| Ligand | Room Temp | 80° C. |
|---|---|---|
| MONOPIC | 0.399 | 0.599 |
| DOTA | 1.268 | 3.616 |
| NO3PIC | ND | 0.0969 |
| NO1A2PIC | 0.0547 | 1.483 |
| NOTALA | 0.0495 | 0.0634 |
| PICAGA* | 0.728 | 0.500 |
| DOTA-DUPA* | 0.900 | 1.421 |

*DUPA-functionalized ligands

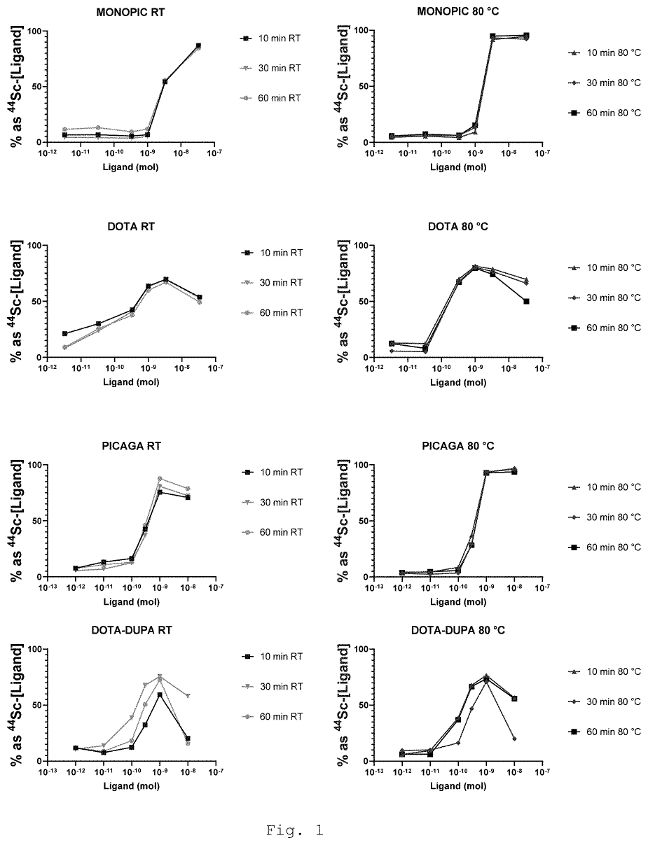

TABLE 3
Time-dependent complexation at 10 nmol at 10, 30, and 60 min
time-points (FIG. 1)

| | Ligand point, temperature, % as [symbol] Sc-Ligand] | | | | | |
|---|---|---|---|---|---|---|
| | t = 10 min | | t = 30 min | | t = 60 min | |
| Ligand | RT | 80° C. | RT | 80° C. | RT | 80° C. |
| MONOPIC | 87 1 | 94.5 | 86.7 | 92.1 | 84.5 | 95.5 |
| DOTA [symbol] | 69.6 | 78.9 | 69.4 | 76.6 | 67 | 74 |
| NO3PIC | 44.3 | 71.1 | 45.4 | 75.1 | 43.4 | 73.3 |
| NO1A2PIC | 38.3 | 94.3 | 48.2 | 97.7 | 53.6 | 93.5 |
| NOTALA | 36.3 | 81.7 | 50.4 | 81.2 | 58.4 | 83.7 |
| P1CAGA* | 70.8 | 96.1 | 72.4 | 96.7 | 78.8 | 93.7 |
| DOTA-DUPA* [symbol] | 59.3 | 76.6 | 75.6 | 70.9 | 72.6 | 73.6 |

*DUPA - functionalized ligands [symbol]
indicates data missing or illegible when filed

Preparation of ⁴⁴Sc-based PSMA tracers for injection. 0.1 mL of sodium citrate (150 mM, pH 4.5) was added to 10 nmol of ligand in 0.1 mL DI water. ⁴⁵ScCl₃ (0.1-0.4 mCi in 500 μL dilute HCl from University of Wisconsin-Madison) was added and the reaction mixture was heated at 80° C. for 30 min. Subsequent reaction monitoring was done by analytical HPLC. The reaction mixture was purified using solid phase extraction (C18 sep pak). Unchelated ⁴⁴Sc is eluted with 100% H₂O, followed by elution of the desired ⁴⁴Sc-complex with a 1:9 EtOH/H₂O mixture. The eluate was collected and concentrated in vacuo and reconstituted in PBS for in vivo injection.

Rₜ: ⁴⁴Sc(PICAGA): stereoisomers elute at 4.5 and 4.6 min; ⁴⁴Sc(DOTA-DUPA): 1.6 min; free ⁴⁴Sc: 0.8 min.

Synthesis of DUPA

Dissolved L-glutamate di-tert-butyl ester hydrochloride and triphosgene into dichloromethane. Add 2.1 molar equivalents of triethylamine. Stirred at −78° C. under N₂ for 2 h. Dissolved H-Glu(Obzl)-otBu.HCl in dichloromethane with 1.3 equivalents triethylamine. Added to reaction mixture. Removed from cold bath stirred overnight. Quench with 0.8 mL HCl. Extract with brine and dry over sodium sulfate. Decanted and removed solvent. Purified using flash chromatography (hexane: EtOAc 1:1). Combined fractions and removed solvent. Added palladium on carbon 10% to flask and dissolved in ethanol. Added balloon with hydrogen gas and stirred overnight. Filtered through PVDF filter and removed solvent under reduced pressure. Dissolved in dichloromethane and added n-carbobenzoxy 1,5-diaminopentane hydrochloride with HBTU. Added 1.1 equivalents of N,N-diisopropylethylamine while stirring. Stirred overnight at room temperature. Removed solvent and purified by reverse-phase flash chromatography. Pooled fractions and removed solvent. Added palladium on carbon 10% to flask and dissolved in ethanol. Added balloon with hydrogen gas and stirred overnight. Filtered through PVDF filter and removed solvent under reduced pressure to result in DUPA ligand with deprotected 1,5-diaminopentane linker.

Synthesis of NOpic and NO2pic

1,4,7-triazacyclononane was dissolved in acetonitrile. K₂CO₃ was added with t-butyl bromopicolinate. Stirred overnight at room temperature and purified by reverse-phase flash chromatography. Pooled fractions and removed solvent under reduced pressure. Dissolved in acetonitrile and K₂CO₁ was added with benzyl bromoacetate. Stirred overnight at room temperature and purified by preparative HPLC. Pooled fractions and removed solvent under reduced pressure. Added palladium on carbon 10% to flask and dissolved in ethanol. Added balloon with hydrogen gas and stirred for 4 h. Purified by preparative HPLC and pooled fractions for NOpic and NO2pic and removed solvent under reduced pressure.

Radiolabeling

Radiochemistry complexes were prepared by dissolving mg of ligand into pH 5.5 ammonium acetate buffer to make a 10 mg/mL solution. 10 mCi of 64CuCl₂. Radiolabeling completion was measured by HPLC-UV/gamma. ⁶⁴Cu-DO2Apic-DUPA and ⁶⁴Cu-NO2pic-DUPA were heated at 60° C. for 20 minutes. The percentage of free ⁵⁴Cu was less than 1% in all three ligands. Ligands were purified by HPLC. The ligand fraction was collected, and solvent was removed under reduced pressure. Purified ligand was re-suspended in 700 uL DPBS for injection.

Cell Binding Assays

26.4 μCi of a standard solution of ⁶⁴Cu-NOpic-DUPA, ⁶⁴Cu-DO2Apic-DUPA, ⁶⁴Cu-NO2pic-DUPA were added to 8.9×105 PSMA positive PC-3 PIP cells and PSMA negative PC-3 Flu cells in 1% FBS/DPBS. Samples were incubated at 37° C. for 60 min. Samples were centrifuged and supernatant was removed. Samples were washed two more times with 1% FBS/DPBS and counted in a γ well-counter for bound activity. Activity of PSMA positive cells was compared to activity of PSMA negative cells to evaluated compound selectivity to the PSMA target.

Cell Culture

PSMA positive PC-3 PIP and PSMA negative PC-3 Flu were obtained from the Case University School of Medicine and grown in RPMI 1640 medium. All media were supplemented with 10% fetal bovine serum and 1% penicillin/streptomycin. Cultures were maintained at 37° C. in 5% carbon dioxide incubator.

Biodistribution and PET/CT Imaging

Subcutaneous tumor xenografts were produced in three groups of four male nude by injecting 7.5×105 cells of PSMA positive PC-3 PIP cells into the right shoulder and PSMA negative PC-3 Flu cells into the left shoulder. Imaging was performed when tumors reached 4-10 mm in diameter. Each group was injected in the tail vein with 146-240 μCi of ⁶⁴Cu-NOpic-DUPA, ⁶⁴Cu-DO2Apic-DUPA, or ⁶⁴Cu-NO2pic-DUPA. PET imaging time points were 30 minutes, 60 minutes and 90 minutes for ⁶⁴Cu-NOpic-DUPA, ⁶⁴Cu-NO2pic-DUPA. Mice injected with ⁶⁴Cu-NO2pic-DUPA were imaged at 60 minutes and 90 minutes. Mice were sacrificed at 2 hours and major were removed and counted in a γ well-counter for activity.

### Example 1. Ligand Design and Screening with ⁴⁴Sc

The coordination chemistry of the Sc(III) ion exhibits parallels to Y(III) and Lu(III), but is dominated by the small ionic radius and a preference for chemically hard donor ions (carboxylates, aliphatic amines). Only a few studies exist on the aqueous chelation chemistry of Sc(III), and radiochemical studies have focused predominantly on the tetraazamacrocycle-derived chelator DOTA. DOTA is not ideal for the complexation of scandium isotopes; [⁴⁴Sc(DOTA)]⁻ only forms quantitatively at 80° C. Furthermore, the strong preference for the formation of kinetically labile “out of cage” complexes with various DOTA-type derivatives has been documented. These findings indicate that chelators with smaller binding cavities are better suited to complex ⁴⁴Sc(III) under mild, low temperature conditions without formation of the problematic “out-of-cage” species.

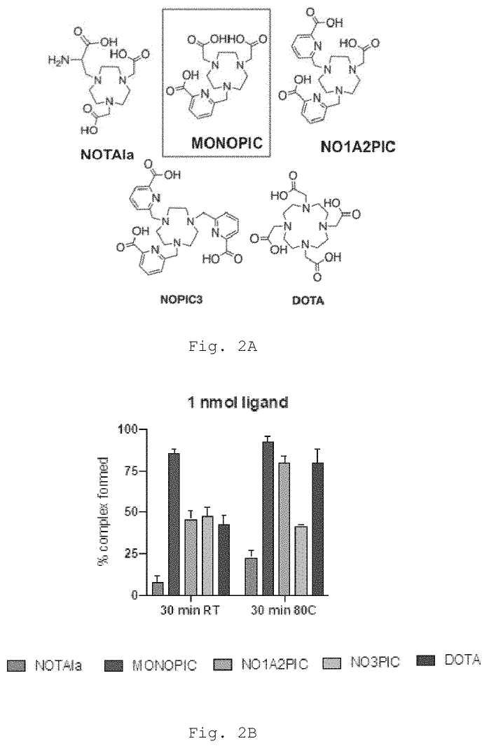

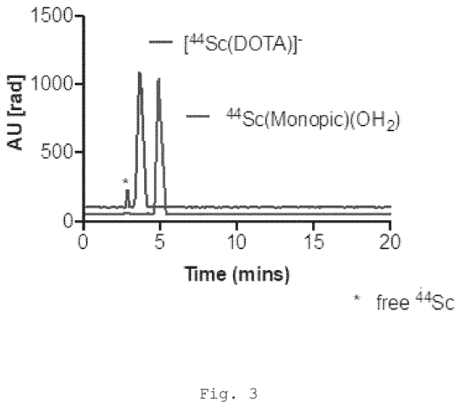

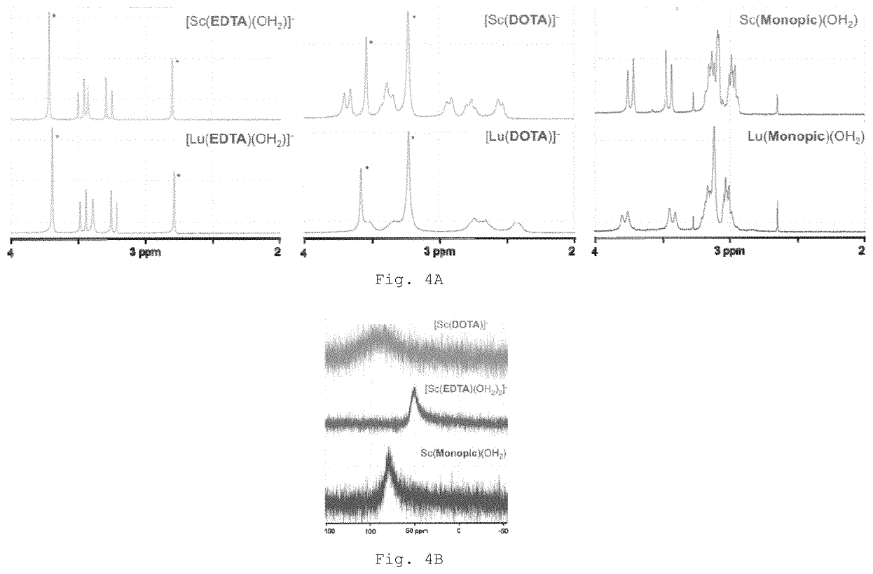

It was hypothesized that an octa- or heptadienoate, triaza-macrocycle based chelator would exhibit rapid complexation kinetics even at room temperature without significant formation of the labile out-of cage complex. We introduced picolinic acid donor arms to increase the number of coordinating donors and impart additional rigidity to formed complexes; picolinate-functionalized triaza-macrocycle chelates have been shown to exhibit high kinetic inertness and slow interconversion of RRRλ- to SSSδ-complex isomers, especially with small lanthanides. We synthesized a chelator library based on picolinic acid-functionalized triaza-cyclononane and tested radiolabeling properties at room temperature and 80° C. with cyclotron-produced ⁴⁴ScCl₃ (0.1 mL reaction volume, 0.25 M, pH 4.5). We identified one lead structure, the heptadienoate chelator, monopic (FIGS. 2A-B), radiolabeling trace FIG. 3). Monopic produces high radiolabeling yields with ⁴⁴Sc at room temperature with ligand quantities of as low as 3 nmol. Assessment of kinetic inertness in presence of 10×EDTA and rat plasma revealed the corresponding complex to exhibit no detectable transchelation. Interestingly, the 8-coordinate, bispicolinate chelator NO1APIC does not coordinate ⁴⁴Sc at room temperature and displays diminished complex inertness in the EDTA-challenge experiment.

### Example 2. Sc(Monopic) Complex Characterization

¹H and ⁴⁵Sc NMR studies were employed to compare the complexation of Sc(III) with DOTA and monopic. The assessment of the dynamic interconversion between δδδ/λλλ isomers informs on structural homology between different M(III) complexes. To estimate isomerism and confirm formation of the kinetically inert in-cage complexes, we carried out ¹H- and ⁴⁵Sc studies with complexes of DOTA and Monopic (FIGS. 4A-B) and 1H studies on the corresponding Lu(III)-complexes. ¹H NMR studies indicate a greater structural homology of the EDTA and Monopic Lu(III) and Sc(III) complexes, with the DOTA complexes differing significantly with respect to rigidity and ring-isomerism at room temperature.

FIG. 4A shows corresponding Lu and Sc complex NMR spectra. [Lu(DOTA)]⁻ indicates significant structural isomerization at room temperature, while the corresponding [Sc(DOTA)]⁻ complex exhibits more clearly resolved structural isomers, but broadening indicates that isomerism may still occur. Sc(Monopic) and Lu(Monopic) exhibit nearly identical ¹H spectra, with a slight broadening of acetate-arm methylenes observed for Sc(Monopic), revealing that isomerization is disfavoured for both complexes. ¹H-NMR spectra of Sc(Monopic) recorded at pH ranges from 1.5-7 show that complexation is favoured even in presence of high proton concentrations and produces one single observed isomer; in contrast, the [Sc(DOTA)]⁻ complex only forms quantitatively above pH 3, while the ¹H NMR spectrum at pH 2 hinting the presence of the protonated, rapidly isomerizing species Sc(DOTAH) which is in accordance with previously published potentiometric data (Figure SX).

In addition to information on the structural dynamics of macrocycle isomerism, ⁴⁵Sc-NMR provides complementary information on shielding of the metal ion by the ligand environment. FIG. 4B shows ⁴⁵Sc-NMR spectra of [Sc (DOTA)]⁻, [Sc (EDTA) (OH₂)₂]⁻ and Sc(Monopic) (OH₂). The corresponding peak chemical shifts were found at 91, 51 and 79 ppm respectively. The Sc-aqua ion produces a sharp signal at 7 ppm, while out-of-cage complexes typically result in ⁴⁴Sc chemical shifts of 20-30 ppm. The pronounced line broadening of [Sc(DOTA)]⁻ indicates structural isomerism, while low relative chemical shift indicates poor shielding of the ⁴⁵Sc-nucleus from solvent molecules. Our ¹H- and ⁴⁵Sc-NMR data indicates that the Sc(Monopic) complex does not exhibit significant metal-centered isomerism; while it is not possible to determine the number of waters bound to the metal ion, NMR-experiments indicate that monopic shields Sc(III) from the solvent environment more efficiently than EDTA, but less efficiently than DOTA, hinting presence of one inner-sphere water. These results are in accordance with NMR-spectroscopic studies on the [Sc (AAZTA) (OH₂)]⁻ complex.

### Example 3. Relative Kinetic Inertness Studies—Functionalization of Monopic

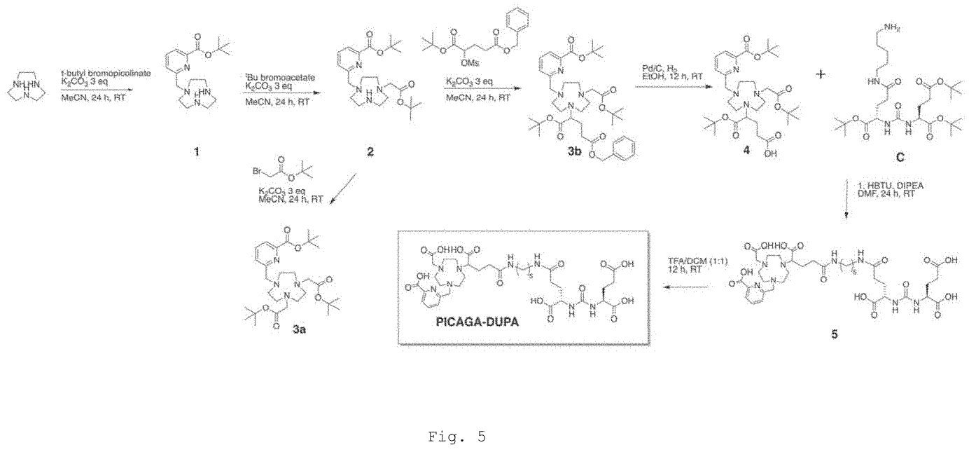

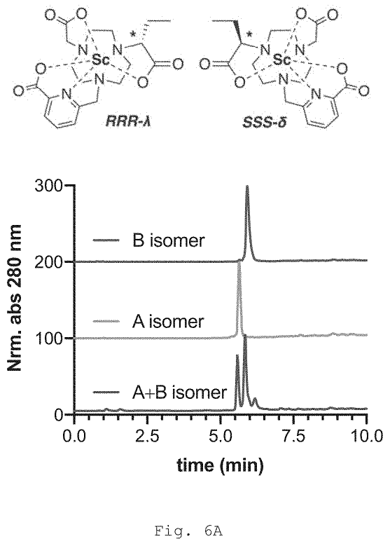

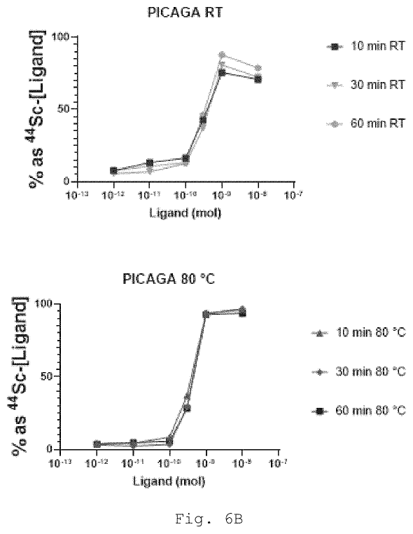

Based on the promising results obtained with the non-functionalized chelate, we synthesized a proof-of-concept bioconjugate, to incorporate a biological targeting vector, 2-[3-(1,3-dicarboxypropyl)ureido]pentanedioic acid (DUPA). DUPA targets the the prostate specific membrane antigen (PSMA) overexpressed in a large fraction of metastasizing prostate cancers; additionally, the limited preclinical and human data has been acquired with PSMA-targeting conjugates and provides a suitable reference to probe the efficacy of a novel conjugate. The monopic ligand was functionalized via a functionalized glutarate in close analogy to the 6-coordinate NODAGA and 8-coordinate DOTAGA chelators, which closely preserve the original ligand donor set. This contrasts the mono-amidation conjugation strategy commonly employed for PSMA-targeted DOTA-conjugates, which converts one of the coordinating carboxylates into an amide. Amide coordination to the Sc(III) ion imparts a significantly altered coordination environment with a greater, not directly predictable impact on the radiolabeling properties and corresponding kinetic inertness. The functionalized monopic derivative, picaga, was synthesized by alkylation of triazacyclononane with benzyl tert-butyl 2-(methylsulfonyloxy)-glutarate, followed by step-wise introduction of tert-butyl-bromoacetate, followed by alkylation with tert-Butyl 6-(bromomethyl)-2-pyridinecarboxylate. Each alkylated intermediate was isolated and purified using reverse-phase preparative HPLC. Following the complete assembly of the orthogonally protected ligand, the benzyl-ester was deprotected and amidated with carboxy-protected, aminopentyl-functionalized DUPA (FIG. 5).

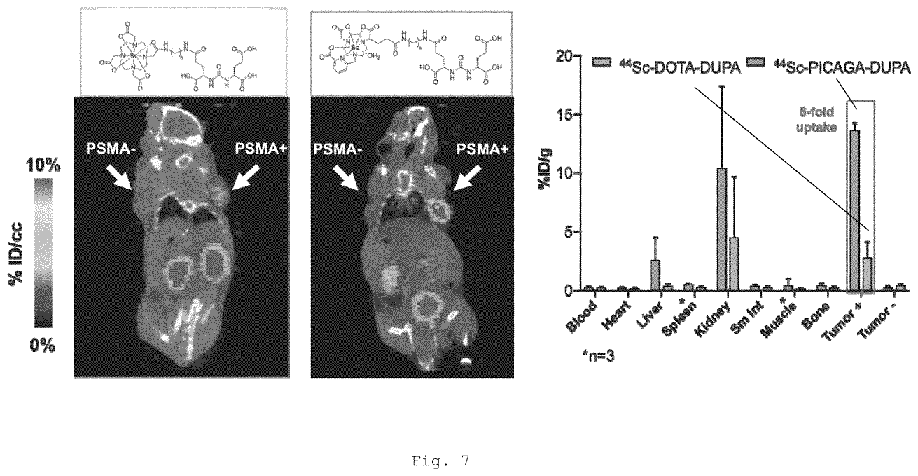

A direct DO3A-monoamide DUPA conjugate analogue was also synthesized. The fully assembled conjugates DOTA-DUPA and picaga-DUPA were fully deprotected and characterized using ¹H-, ¹³C-NMR and mass spectrometry. Following complete characterization, the non-radioactive scandium complexes were formed and characterized using HPLC-analysis. As the glutarate arm is introduced as the pure S-entantiomer, where the alpha carbon retains the chiral alpha carbon and subsequently introduces chirality to the ligand. Upon coordination to Sc(III) or Lu(III), this results the formation of stable diasteromers with distinct HPLC-retention times (FIG. 6A-B). The Sc(III) diastereomers can be chromatographically separated and do not interconvert even after 12 hours, exemplifying the high kinetic inertness of the corresponding diastereomer with respect isomerization

### Example 4. Complexation with ⁴⁴Sc

Both DOTA and picaga-DUPA conjugates were subjected to radiochemical complexation experiments in dependence of temperature and time. ⁴⁴Sc-picaga-DUPA forms high radiochemical yields with 1 nmol of conjugate: 78% complexation is achieved at room temperature and 96% at 80° C. within only 10 minutes. This compares favorably to radiolabeling of ⁴⁴Sc with DOTA-conjugates at 95° C. The radiochemical complexes formed also exhibit characteristic diastereomer formation, with a major and minor stereoisomer separated by 0.1 minute retention time.

### Example 5. In Vivo Imaging and Biodistribution Experiments

To assess the in vivo behavior of ⁴⁴Sc-picaga-DUPA, we administered the radiolabeled compound to mice bearing PSMA+ and PSMA− tumor xenografts on the right and the left flank respectively. As a reference, the corresponding ⁴⁴Sc-DOTA-DUPA complex was also synthesized and used as a reference compound. Mice were imaged at 90 minutes post injection using PET-CT and subsequently sacrificed for a 120 minute biodistribution.

The animal studies reveal that both ⁴⁴Sc-picaga-DUPA and ⁴⁴Sc-DOTA-DUPA exhibit excellent properties for in vivo imaging of PSMA+ cancer xenografts; however, the uptake achieved with ⁴⁴Sc-picaga-DUPA was 6 times greater than what was observed for the corresponding DOTA conjugate (FIG. 7). The increased lipophilic character of the ⁴⁴Sc-picaga-DUPA conjugate results in slower blood clearance and subsequently greater amount of compound being delivered to the tumor tissue. The increased lipophilicity is further evidenced by the slightly enhanced liver uptake in comparison with the DOTA conjugate.

### Example 6. Call Binding Assay

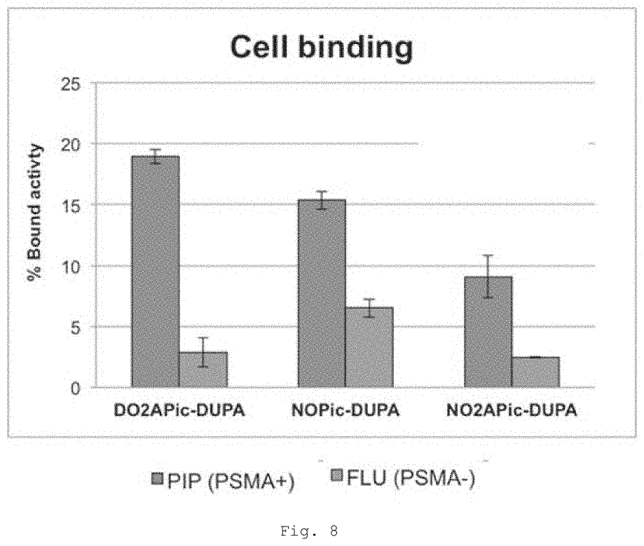

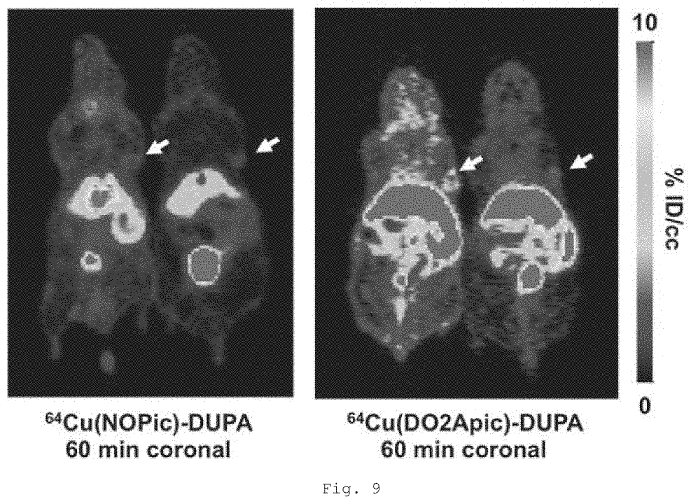

All ligands demonstrated selective binding to PSMA positive PC-3 PIP cells over PSMA negative PC-3 Flu cells (FIG. 8).

### Example 7. In Vivo Performance

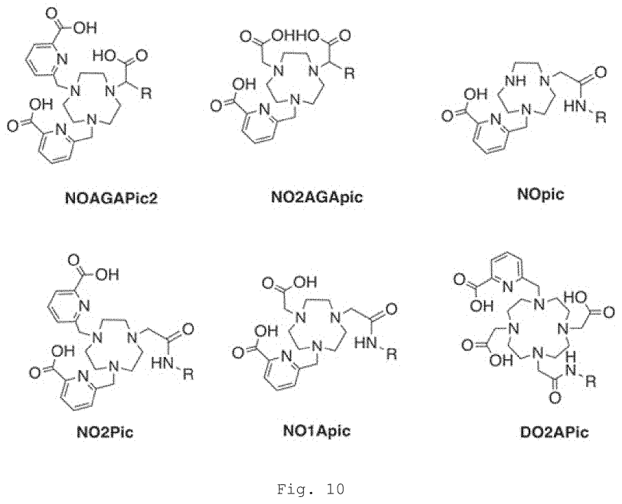

All three compounds were injected into nude mice bearing PSMA+ and PSMA− xenograft tumors (FIG. 9). All three compounds were used to successfully image PSMA+ tumors and dynamic PET imaging shows rapid renal clearance and liver uptake. Biodistribution is consistent with dynamic PET imaging and indicates enhanced, target specific accumulation of ⁶⁴Cu(DO2Apic)-DUPA, and ⁶⁴Cu (NOpic-DUPA) in PSMA− expressing xenografts.

### Example 8. Selective Imaging Probes

An additional aspect of the invention provides compounds with imaging agents that can be selectively taken up by prostate cancer cells but not normal cells. The technology provides superior noninvasive cancer detection by utilizing prostate specific membrane antigen (PSMA) which is overexpress in cancer cells. Relative to normal cells, local probe concentrations in prostate cancer cells are retained substantially higher over longer time period.

### Example 9: Cancer Cell Imaging in a Subject

The compound of the present invention is administered to a subject afflicted with prostate cancer. The targeting moiety in the compound binds to the cancer cells in the subject. The cancer cells in the subject are detected using a molecular imaging device based on the location of the compound in the subject and an image of the prostate cancer cells is obtained.

### DISCUSSION

The concept of personalized medicine, where patient treatment is performed according to an individually tailored treatment regime, is an emerging clinical management paradigm. In nuclear medicine, this approach is realized by exploiting diagnostic techniques, such as non-invasive imaging by means of Positron Emission Tomography (PET) and Single Photon Emission Computed Tomography (SPECT), followed by individualized radiotherapeutic treatment. If the same molecular targeting vector is labeled with the diagnostic and the therapeutic radionuclide and utilized for sequential imaging and treatment, the approach is considered theranostic. Ideally, the employed radionuclides represent a matched pair, where both are radioisotopes of the same chemical element; however, only few elements have isotopes with suitable emission properties for this purpose, thus clinical applications have focused on perceived chemical homologues such as ⁶⁸Ga(III) for PET imaging and ¹⁷⁷Lu(III) for subsequent radiotherapy. The vast differences in coordination chemistry of these two metal ions result in large discrepancies with respect to chemical and biological behavior: The resulting differences of ⁶⁸Ga and ¹⁷⁷Lu-complexes in lipophilicity, receptor bindingaffinity and in vivo biodistribution are well documented. The ability of ⁶⁸Ga-complexes to accurately predict distribution and behavior of the corresponding ¹⁷⁷Lu-compounds is limited and can lead to incorrect dose calculations.

There is a clear need to develop theranostic isotope pairs where diagnostic imaging is directly, accurately and reliably predictive of therapy. The half-life of ⁴⁴Sc of 3.97 h is almost 4-fold longer than that of ⁶⁸Ga (t_{1/2}=68 min) and, hence, allows use with biomolecules with slower kinetics and shipping of the isotope over long distances. The recently increased availability of ⁴⁴Sc has initiated a number of preclinical in vitro and in vivo studies with DOTA-conjugated biomolecules. The emission properties of ⁴⁷Sc (Eβ⁻ avg=162 keV, tu, =80.4 h, (Table 4) are comparable to the clinically established ¹⁷⁷Lu (Eβ⁻avg=134 keV, t_{1/2}=159.6 h). In analogy to ¹⁷⁷Lu, the decay of ⁴⁷Sc is characterized by the co-emission of γ-rays with an ideal energy (Eγ=159 keV) for SPECT imaging.

TABLE 4
Properties of scandium isotopes in comparison with the
currently used_theranostic pair ⁶⁸Ga/¹⁷⁷Lu

| Isotope | | |
|---|---|---|
| (half-life) | Emission properties | Source of isotope |
| ⁶⁸Ga (1.1 h) | 830 keV (Eβ⁺_{avg}) | Generator: ⁶⁸Ge/⁶⁸Ga |
| ⁴⁴Sc (4 h) | 632 keV (Eβ⁺_{avg}) | Generator: ⁴⁴Ti/⁴⁴Sc |
| | | Cyclotron (11 MeV): |
| | | ⁴⁴Ca(p, n)⁴⁴Sc |
| ⁴⁷Sc (80.4 h) | 162 keV (Eβ⁻_{avg}) | Cyclotron (24 MeV): |
| | | ⁵⁰Ti(p, α)⁴⁷Sc |
| ¹⁷⁷Lu (159.6 h) | 132 keV (Eβ⁻_{avg}) | Reactor: |
| | | ¹⁷⁶Lu(n, γ)¹⁷⁷Lu |

While DOTA is considered the gold standard for the formation of stable scandium(III)-chelates, the slow complexation kinetics require long reaction times at 70-95° C., which is incompatible with the labeling of antibodies and other biomolecules. Hexadentate and acyclic chelators coordinate Sc(III) more rapidly than DOTA, but with a markedly reduced stability of the corresponding chelates, resulting in rapid release of Sc-isotopes in vitro and in vivo. In order to establish the scandium theranostic isotope pair as a clinical option for diagnosis and therapy of disease, the thorough understanding of the aqueous coordination and radiochemistry of scandium(III) is required. Here, we introduce a lanthanide chemistry inspired, rational ligand design to develop an optimal bifunctional chelator for scandium isotopes which fulfills the following criteria: 1) Radiolabeling is achieved at room temperature and various pH conditions (2-6), to enable kit formulations and the labeling of temperature-sensitive biomolecules 2) The chelator disfavors the formation of an out-of-cage complex to prevent the formation of kinetically labile, intermittently formed complex species 3) The formed complex is kinetically inert to transchelation in vitro and in vivo and 4) Functionalization of the ligand is feasible without negatively impacting radiolabeling properties or the kinetic inertness of the formed complex.

⁴⁴Scandium and ⁶⁴copper have recently emerged as an attractive, short-lived, PET isotopes with a matched radiotherapeutic isotope for therapy (⁴⁷Sc, ⁶⁷Cu). As described herein, novel, modular chemical compositions with high affinity to copper and scandium radioisotopes and a freely functionalizable moiety have been synthesized and appended to small molecules targeting the prostate specific membrane antigen (PSMA). These constructs are suitable to kit-type formulations for single-step radiochemical synthesis of diagnostic and therapeutic entities with PSMA-targeting vectors, but can be expanded to applications using other targeting vectors of peptidic/small molecule, protein or antibody nature.

Embodiments of the invention described herein allows for the radiolabeling of PSMA-targeting small molecules with an array of radioactive isotopes suitable for imaging and therapy approaches. This renders the compositions and methods described herein superior to currently commercially available ¹⁸F, ⁶⁸Ga and ¹⁷⁷Lu-based tracers.

Embodiments of the invention disclosed herein provide bifunctional chelators (FIG. 10) suitable for the ⁴⁴Sc/⁴⁴Sc and ⁶⁴Cu/⁶⁷Cu isotope pairs, which allow facile attachment to targeting vectors to furnish small-molecular theranostic tracers in a simple, one-step radiolabeling procedure. Our first application is focused on generating tracers for prostate cancer, amenable to kit-formulations requiring a simple one-step, one-pot radiolabeling protocol, optimal for radiopharmacies to improve diagnostic and therapy workflow. The bifunctional chelator technology is widely applicable to other peptide, protein- and even antibody-based targeted diagnostics and therapeutics.

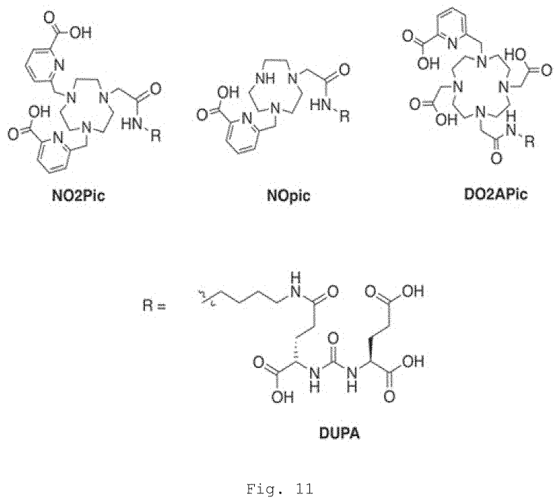

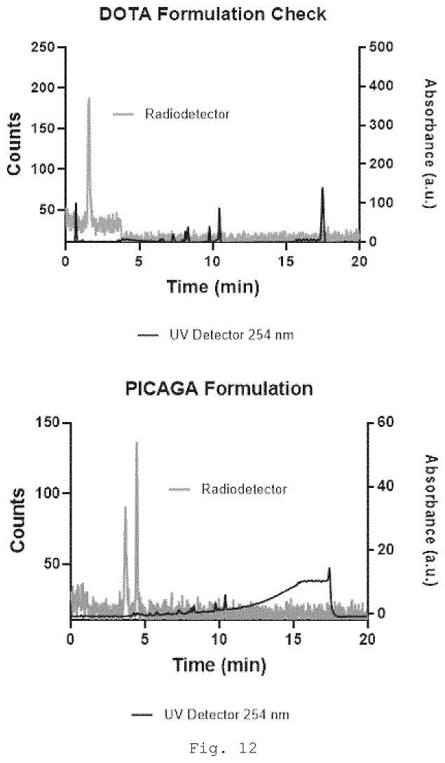

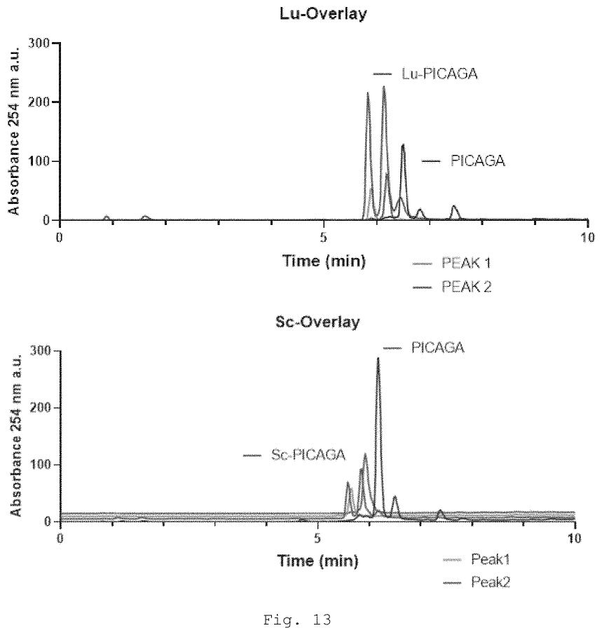

Summary of results with PSMA-targeting conjugates DO2Apic-DUPA, NO2pic-DUPA and NOpic-DUPA (FIG. 11).
Radiolabeling Copper-64 labeling efficiency is rapid and can be carried out at room temperature
Radiolabeling was 99% for NOpic-DUPA and NO2pic-DUPA after 5 minutes at room temperature.
Labeling was 86t for DO2Apic-DUPA after 5 minutes at room temperature and 99% after 30 minutes at 60° C.
All compounds were successfully purified using HPLC prior to in vivo administration.

### REFERENCES

1. Siegel, R. L. et al. Cancer statistics, 2016. CA: a cancer journal for clinicians 2016, 66 (1), 7-30.
2. Moreira, D. M. et al. Predicting time from metastasis to overall survival in castration-resistant prostate cancer: results from SEARCH. Clin. Genitourin. Cancer 2017, 15 (1), 60-66. e2.
3. Meijs, W. E. et al. Zirconium-labeled monoclonal antibodies and their distribution in tumor-bearing nude mice. J. Nucl. Med. 1997, 38 (1), 112.
4. Kahn, D. et al. (111) Indium-capromab pendetide in the evaluation of patients with residual or recurrent prostate cancer after radical prostatectomy. J. Urol. 1998, 159 (6), 2041-2047.
5. Mease, R. C. et al. PET imaging in prostate cancer: focus on prostate-specific membrane antigen. Curr. Top. Med. Chem. 2013, 13 (8), 951-962.
6. Davis, M. I. et al. Crystal structure of prostate-specific membrane antigen, a tumor marker and peptidase. Proc. Nat. Acad. Sci. 2005, 102 (17), 5981-5986.
7. Hillier, S. M. et al. Preclinical evaluation of novel glutamate-urea-lysine analogues that target prostate-specific membrane antigen as molecular imaging pharmaceuticals for prostate cancer. Cancer Res. 2009, 69 (17), 6932-6940.
8. Zechmann, C. M. et al. Radiation dosimetry and first therapy results with a 124I/131I-labeled small molecule (MIP-1095) targeting PSMA for prostate cancer therapy. Eur. J. Nucl. Med. Mol. Imaging 2014, 41 (7), 1280-1292.
9. Szabo, Z. et al. Initial evaluation of [¹⁸F] DCFPyL for prostate-specific membrane antigen (PSMA)-targeted PET imaging of prostate cancer. Mol. Imaging. Biol. 2015, 17 (4), 565-574.
10. Rowe, S. P. et al. PSMA-based [¹³F] DCFPyL PET/CT is superior to conventional imaging for lesion detection in patients with metastatic prostate cancer. Mol. Imaging. Biol. 2016, 18 (3), 411-419.
11. Schmitt, M. et al. Promising prospects for 44Sc-/47Sc-based theragnostics: application of 47Sc for radionuclide tumor therapy in mice. J. Nucl. Med. 2014, 55 (10), 1658-1664.
13. van der Meulen, N. P. et al. Cyclotron production of 44Sc: from bench to bedside. Nucl. Med. Biol 2015, 42 (9), 745-751.
14. Radchenko, V. et al. Proton-induced production and radiochemical isolation of ⁴⁴Ti from scandium metal targets for ⁴⁴Ti/⁴⁴Sc generator development. Nucl. Med. Biol 2017, 50, 25-32.
15. Umbricht, C. A. et al. ⁴⁴Sc-PSMA-617 for radiotheragnostics in tandem with ¹⁷⁷Lu-PSMA-617-preclinical investigations in comparison with ⁶⁸Ga-PSMA-11 and ⁵⁸Ga-PSMA-617. EJNMMI research 2017, 7 (1), 9.

1. A compound having the structure: 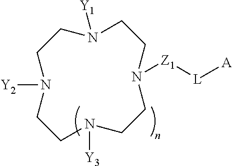
wherein
n is 0 or 1;
Y₁, Y₂ and Y₃ are each, independently, —H, alkylheteroaryl, alkyl-CO₂H, alkylaryl-CO₂H, alkylheteroaryl-CO₂H, alkyl-CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N (alkylaryl-OH) (alkyl-CO₂H), alkyl-N(alkylheteroaryl-OH) (alkyl-CO₂H), alkyl-P(O) (OH)₂, alkylaryl-P(O) (OH)₂ and alkylheteroaryl-P(O) (OH)₂,
wherein each occurrence of R₄ is, independently, —H, alkyl, alkenyl, alkynyl, alkyl-aryl, alkyl-heteroaryl, aryl, heteroaryl, alkyl-CF₃ or —Si(alkyl)₃;
Z₁ is 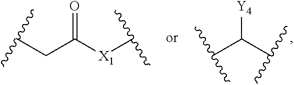
wherein X₁ is NH, O or S, and
Y₄ is —CO₂H, —CO₂R₅, aryl-CO₂H, heteroaryl-CO₂H, aryl-CO₂R₅ or heteroaryl-CO₂R₅,
wherein each occurrence of R₅ is, independently, —H, alkyl, alkenyl, alkynyl, alkyl-aryl, alkyl-heteroaryl, aryl, heteroaryl, alkyl-CF₃ or —Si (alkyl)₃;
A is a targeting moiety; and
L is a chemical linker,
or a pharmaceutically acceptable salt of the compound.

2-4. (canceled)

5. The compound of claim 1, wherein the targeting moiety A is trastuzumab, bombesin, somatostatin or 2-[3-(1,3-dicarboxypropyl)ureido]pentanedioic acid (DUPA) or a derivative or fragment thereof.

6-8. (canceled)

9. The compound of claim 1, wherein the chemical linker L is an alkyl, alkenyl, alkynyl, alkylether, alkylthioether, alkylamino, alkylamido, alkylester, alkylaryl, alkylheteroaryl, aryl, heteroaryl, a natural amino acid, an unnatural amino acid, a disulfide or thioether containing linker or combinations thereof.

10-11. (canceled)

12. The compound of claim 1 having the structure:

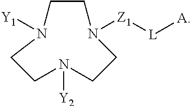

13-14. (canceled)

16. The compound of claim 1 having the structure:

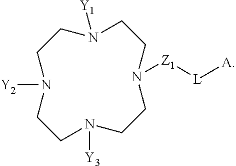

17-20. (canceled)

21. A metal complex comprising the compound of claim 1, wherein the compound coordinates to a metal.

22. (canceled)

23. The metal complex of claim 21 having the structure: 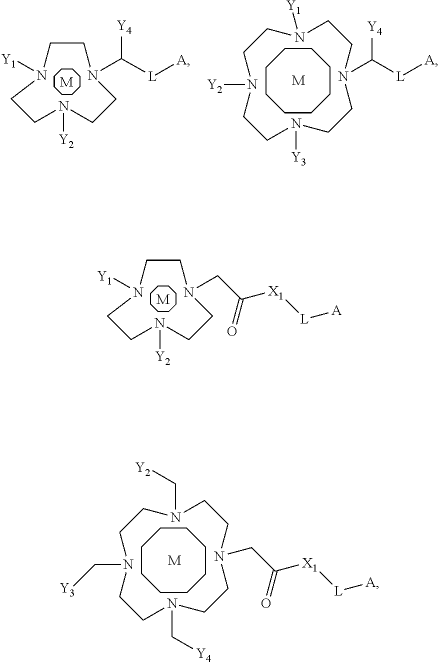
or a pharmaceutically salt thereof.

24. (canceled)

25. A method of detecting target cells in a subject comprising administering an effective amount of the metal complex of claim 21 to the subject, and imaging the subject with a molecular imaging device to detect the metal complex in the subject.

26-27. (canceled)

28. A method of detecting the presence of target cells in a subject which comprises determining if an amount of the metal complex of claim 21 or a pharmaceutically acceptable salt thereof, is present in the subject at a period of time after administration of the metal complex to the subject, thereby detecting the presence of the target cells based on the amount of the metal complex determined to be present in the subject.

29. (canceled)

30. The compound of claim 1
where A has the structure: 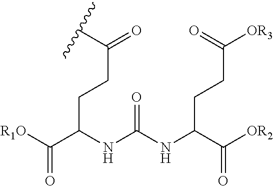
wherein
R₁, R₂ and R₃ are each, independently, —H, alkyl, alkenyl, alkynyl, alkyl-aryl, alkyl-heteroaryl, aryl, heteroaryl, alkyl-CF₃ or —Si(alkyl)₃.

31-32. (canceled)

33. The compound of claim 30, wherein R₁, R₂ and R₃ are each H.

34-35. (canceled)

36. The compound of claim 1 having the structure: 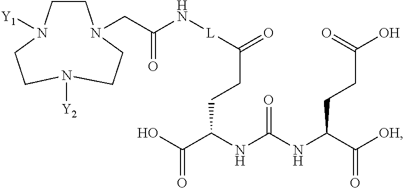
wherein
Y₁ is —H, 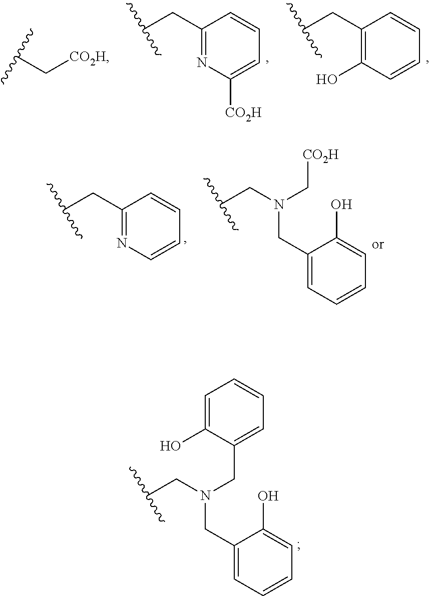
Y₂ is —H, 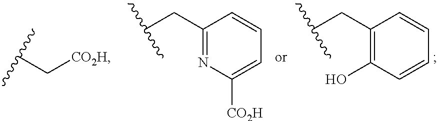 [SMILES: *CC(=O)O ; *Cc1cccc(C(=O)O)n1 ; *Cc1ccccc1O]
L is alkyl-NH,
or a pharmaceutically acceptable salt of the compound.

37. The compound of claim 1 having the structure: 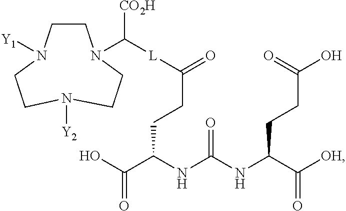
wherein
Y₁ is —H, 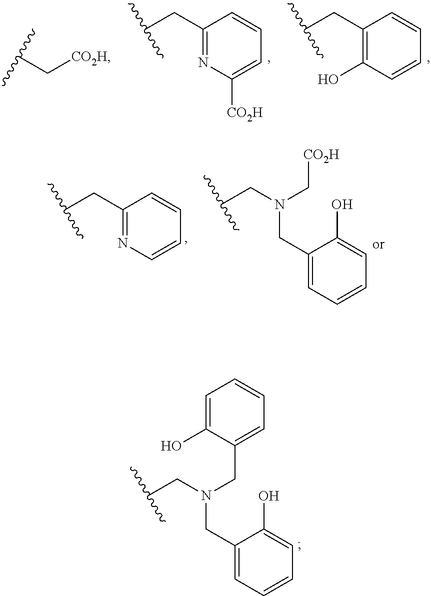
Y₂ is —H, 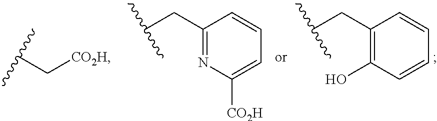 [SMILES: *CC(=O)O ; *Cc1cccc(C(=O)O)n1 ; *Cc1ccccc1O]
L is alkyl-C(O)NH-alkyl-NH,
or a pharmaceutically acceptable salt of the compound.

38. The compound of claim 1 having the structure: 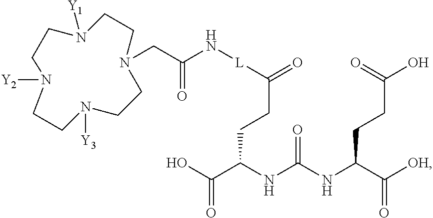
wherein
Y₁ is —H, 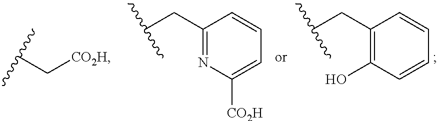 [SMILES: *CC(=O)O ; *Cc1cccc(C(=O)O)n1 ; *Cc1ccccc1O]
Y₂ is —H, 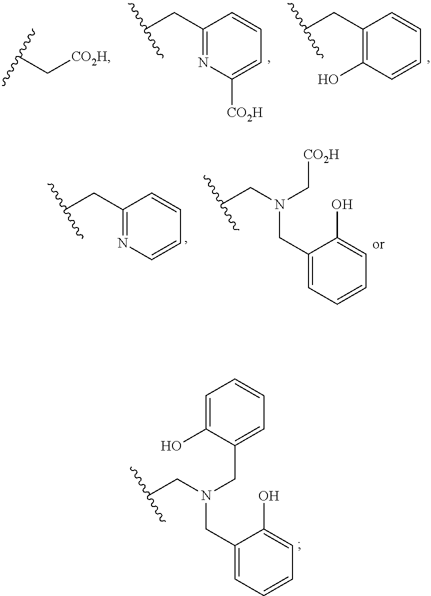
Y₃ is —H, 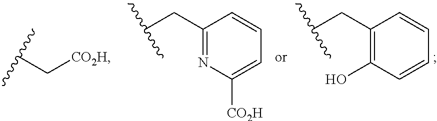 [SMILES: *CC(=O)O ; *Cc1cccc(C(=O)O)n1 ; *Cc1ccccc1O]
L is alkyl-NH,
or a pharmaceutically acceptable salt of the compound.

39. The compound of claim 1 having the structure: 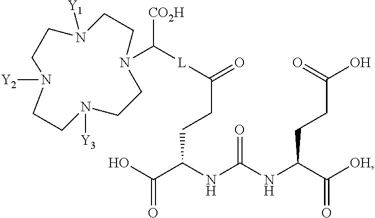
wherein
Y₁ is —H, 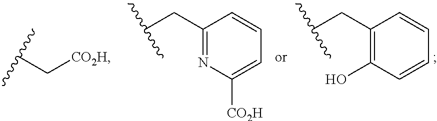 [SMILES: *CC(=O)O ; *Cc1cccc(C(=O)O)n1 ; *Cc1ccccc1O]
Y₂ is —H, 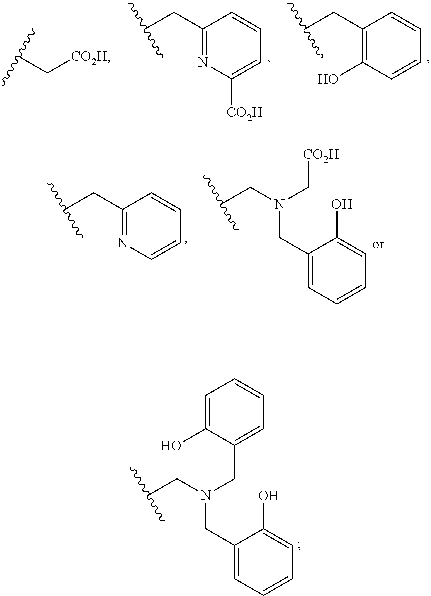
Y₃ is —H, 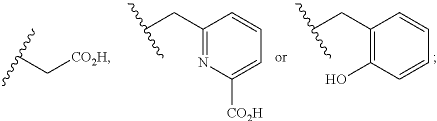 [SMILES: *CC(=O)O ; *Cc1cccc(C(=O)O)n1 ; *Cc1ccccc1O]
L is an alkyl-C(O)NH-alkyl chemical linker,
or a pharmaceutically acceptable salt of the compound.

40. The compound of claim 1 having the structure: 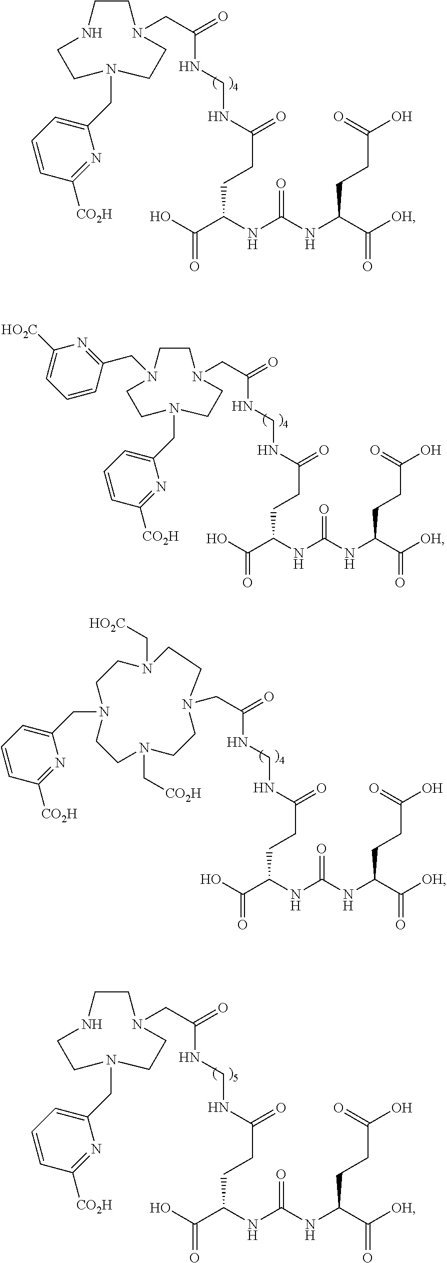 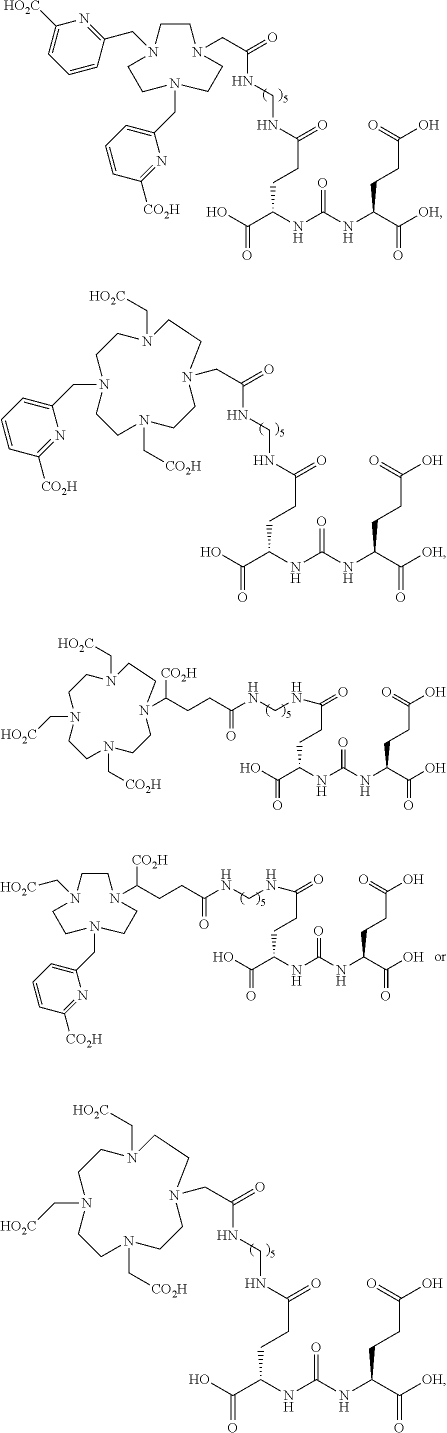
or a pharmaceutically salt thereof.

41. (canceled)

42. A pharmaceutical composition comprising the compound of claim 1 and a pharmaceutically acceptable carrier.

43-44. (canceled)

45. The metal complex of claim 21 having the structure: 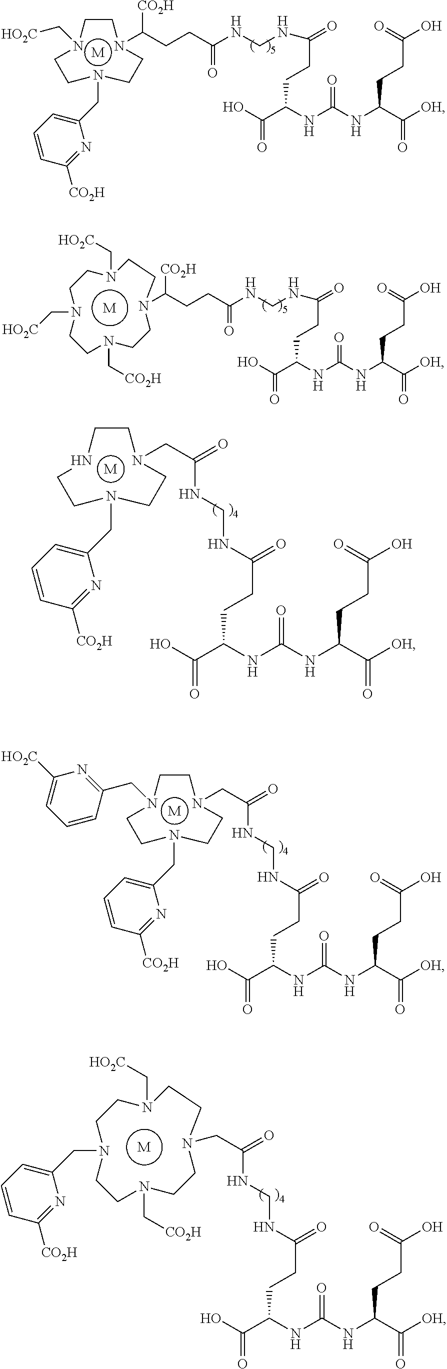 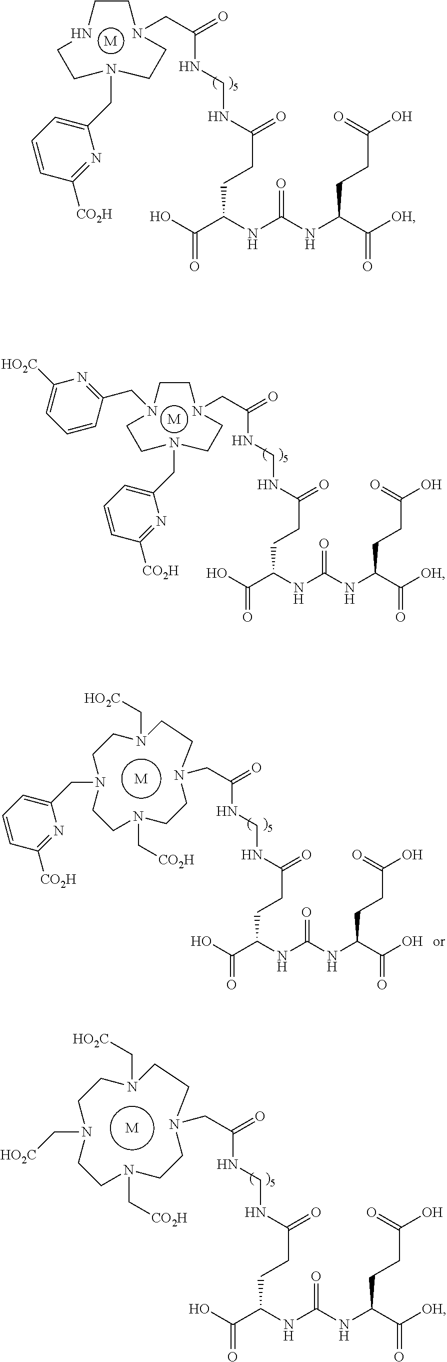
or a pharmaceutically salt thereof.

46-47. (canceled)

48. A method of imaging prostate cancer cells in a subject comprising:
1) administering to the subject an effective amount of the metal complex of claim 21 or a pharmaceutically acceptable salt thereof,
wherein the compound specifically accumulates at prostate cancer cells in the subject;
3) detecting in the subject the location of the metal complex; and
4) obtaining an image of the cancer cells in the subject based on the location of the metal complex.

49. (canceled)

50. A method of reducing the size of a prostate tumor or of inhibiting proliferation of prostate cancer cells comprising contacting the tumor or cancer cells with the metal complex claim 21 or a pharmaceutically acceptable salt thereof, so as to thereby reducing the size of the tumor or inhibit proliferation of the cancer cells.
